(19) United States
(12) Patent Application Publication
(10) Pub. No.: US 2011/0082185 A1
(43) Pub. Date: Apr. 7, 2011

(54) CANCER-TESTIS GENE SILENCING AGENTS AND USES THEREOF

(75) Inventors: Otavia L. Caballero, Fairfield, CT (US); Tzeela Cohen, New York, NY (US); Andrew John George Simpson, Westport, CT (US)

(73) Assignee: Ludwig Institute for Cancer Research Ltd., New York, NY (US)

(21) Appl. No.: 12/678,535

(22) PCT Filed: Sep. 16, 2008
(86) PCT No.: PCT/US08/10797

§ 371 Date: Dec. 6, 2010

Related U.S. Application Data
(60) Provisional application No. 60/994,244, filed on Sep. 17, 2007.
(60) Provisional application No. 61/002,487, filed on Nov. 9, 2007.

(51) Int. Cl. A61K 31/7088; C07H 21/02; C12N 5/02; A61P 35/00

(52) U.S. Cl. 514/44 A; 536/24.5; 435/375

(57) ABSTRACT

The invention relates to methods, formulations and kits useful for inhibiting cancer cell viability, invasion, or migration.

### RELATED APPLICATIONS

This application claims the benefit under 35 U.S.C. §119(e) of U.S. provisional application No. 60/994,244, filed Sep. 17, 2007, and U.S. provisional application No. 61/002,487, filed Nov. 9, 2007, the disclosures of which are incorporated herein by reference.

### FIELD OF THE INVENTION

The invention relates to methods, formulations and kits useful for inhibiting cancer cell viability, invasion, or migration.

### BACKGROUND OF THE INVENTION

Malignant tumors are characterized by a tendency for sustained growth and an ability to spread or metastasize to distant locations. If left untreated, malignant tumors will ultimately result in death of an individual with cancer. Metastasis associated with malignant tumors involves an array of basic cellular activities that include invasion, migration, and extracellular matrix attachment. While each of these metastatic activities presents an opportunity for therapeutic intervention to treat cancer, they are also important in normal cells, for example, cells of the immune system. Consequently, therapeutic modalities that affect cells indiscriminately could be deleterious. Thus, a key objective of cancer research is to develop cancer cell specific therapeutic strategies for inhibiting metastasis and/or viability of malignant tumors.

### SUMMARY OF INVENTION

The invention disclosed herein relates to the development and use of siRNA molecules of 27 nucleotides in length (“27 mers”) that specifically inhibit the expression of members of the cancer-testis antigens (CT) family, specifically, MAGEA, SSX, CTAG1B, MAGEC1, MAGEC2, XAGE1 and GAGE. The invention further relates to the discovery that inhibition of the expression of certain cancer-testis antigen genes (e.g., SSX, XAGE1, and GAGE) causes reduction in migration, invasion, colony formation, and viability (e.g., survival) specifically in cancer cells (e.g., melanoma. prostate, and lung cancer cells). In some aspects, the invention related to methods for inhibiting expression of MAGEA, SSX, CTAG1B, MAGEC1, MAGEC2, XAGE1 and GAGE in cells (e.g., cancer cells).

According to some aspects of the invention, isolated small interfering nucleic acids are provided. In some embodiments, the isolated small interfering nucleic acids comprise a nucleic acid consisting of the sequence set forth in SEQ ID NO. 2. In some embodiments, the isolated small interfering nucleic acids comprise a nucleic acid consisting of the sequence set forth in SEQ ID NO. 4. In some embodiments, the isolated small interfering nucleic acids comprise a nucleic acid consisting of the sequence set forth in SEQ ID NO. 6. In some embodiments, the isolated small interfering nucleic acids comprise a nucleic acid consisting of the sequence set forth in SEQ ID NO. 8. In some embodiments, the isolated small interfering nucleic acids comprise a nucleic acid consisting of the sequence set forth in SEQ ID NO. 10. In some embodiments, the isolated small interfering nucleic acids comprise a nucleic acid consisting of the sequence set forth in SEQ ID NO. 12. In some embodiments, the isolated small interfering nucleic acids comprise a nucleic acid consisting of the sequence set forth in SEQ ID NO. 14. In some embodiments, the isolated small interfering nucleic acids comprise a nucleic acid consisting of the sequence set forth in SEQ ID NO. 22. In some embodiments, the isolated small interfering nucleic acids comprise a nucleic acid consisting of the sequence set forth in SEQ ID NO. 24. In some embodiments, the isolated small interfering nucleic acids comprise a nucleic acid consisting of the sequence set forth in SEQ ID NO. 26. In some embodiments, the isolated small interfering nucleic acids comprise a nucleic acid consisting of the sequence set forth in SEQ ID NO. 28.

In some embodiments, the isolated small interfering nucleic acids have a sense strand consisting of the sequence set forth in SEQ ID NO. 1 and an antisense strand consisting of the sequence set forth in SEQ ID NO. 2. In certain embodiments, the isolated small interfering nucleic acids have a sense strand consisting of the sequence set forth in SEQ ID NO. 3 and an antisense strand consisting of the sequence set forth in SEQ ID NO. 4. In certain embodiments, the isolated small interfering nucleic acids have a sense strand consisting of the sequence set forth in SEQ ID NO. 5 and an antisense strand consisting of the sequence set forth in SEQ ID NO. 6. In certain embodiments, the isolated small interfering nucleic acids have a sense strand consisting of the sequence set forth in SEQ ID NO. 7 and an antisense strand consisting of the sequence set forth in SEQ ID NO. 8. In certain embodiments, the isolated small interfering nucleic acids have a sense strand consisting of the sequence set forth in SEQ ID NO. 9 and an antisense strand consisting of the sequence set forth in SEQ ID NO. 10. In certain embodiments, the isolated small interfering nucleic acids have a sense strand consisting of the sequence set forth in SEQ ID NO. 11 and an antisense strand consisting of the sequence set forth in SEQ ID NO. 12. In certain embodiments, the isolated small interfering nucleic acids have a sense strand consisting of the sequence set forth in SEQ ID NO. 13 and an antisense strand consisting of the sequence set forth in SEQ ID NO. 14. In certain embodiments, the isolated small interfering nucleic acids have a sense strand consisting of the sequence set forth in SEQ ID NO. 21 and an antisense strand consisting of the sequence set forth in SEQ ID NO. 22. In certain embodiments, the isolated small interfering nucleic acids have a sense strand consisting of the sequence set forth in SEQ ID NO. 23 and an antisense strand consisting of the sequence set forth in SEQ ID NO. 24. In certain embodiments, the isolated small interfering nucleic acids have a sense strand consisting of the sequence set forth in SEQ ID NO. 25 and an antisense strand consisting of the sequence set forth in SEQ ID NO. 26. In certain embodiments, the isolated small interfering nucleic acids have a sense strand consisting of the sequence set forth in SEQ ID NO. 27 and an antisense strand consisting of the sequence set forth in SEQ ID NO. 28.

In some embodiments, the isolated small interfering nucleic acids are 27-mer siRNAs.

In some embodiments, the isolated small interfering nucleic acids are short-hairpin RNAs.

According to other aspects of the invention, compositions comprising any of the foregoing isolated small interfering nucleic acids are provided. In some embodiments, the compositions further comprise a transfection reagent.

According to another aspect of the invention, methods for inhibiting expression of a cancer testis antigen in a cell are provided. In some embodiments, the methods involve contacting the cell with a composition comprising any of the foregoing isolated small interfering nucleic acids. In some embodiments, the contacting results in uptake of the isolated small interfering nucleic acid in the cell.

According to another aspect of the invention, pharmaceutical formulations are provided. In some embodiments, the pharmaceutical formulations comprise: (i) any of the foregoing isolated small interfering nucleic acids and (ii) a pharmaceutically acceptable carrier.

According to another aspect of the invention, pharmaceutical kits are provided. In some embodiments, the pharmaceutical kits comprise (i) a container(s) housing a pharmaceutical formulation that comprises: any of the foregoing isolated small interfering nucleic acids and a pharmaceutically acceptable carrier, and (ii) instructions for administering the pharmaceutical formulation to an individual.

According to another aspect of the invention, reagent kits are provided. In some embodiments, the reagent kits comprise: (i) a container housing a composition comprising any of the foregoing isolated small interfering nucleic acids, (ii) instructions for transfecting a cell with the small interfering nucleic acid, and optionally (iii) a container housing a transfection reagent.

According to another aspect of the invention, methods for inhibiting viability, invasion, colony formation, and/or migration of a cancer cell are provided. In some embodiments, the methods involve contacting the cancer cell with an effective amount of a molecule capable of inhibiting expression of XAGE, a molecule capable of inhibiting expression of GAGE, and/or a molecule capable of inhibiting expression of SSX. In certain embodiments, the molecule capable of inhibiting expression of XAGE is or encodes a small interfering nucleic acid capable of inhibiting expression of XAGE, the molecule capable of inhibiting expression of GAGE is or encodes a small interfering nucleic acid capable of inhibiting expression of GAGE, and/or the molecule capable of inhibiting expression of SSX is or encodes a small interfering nucleic acid capable of inhibiting expression of SSX. In specific embodiments, the small interfering nucleic acid capable of inhibiting expression of GAGE comprises a nucleic acid sequence consisting of SEQ ID NO. 2 or SEQ ID NO. 4. In specific embodiments, the small interfering nucleic acid capable of inhibiting expression of XAGE comprises a nucleic acid sequence consisting of SEQ ID NO. 6 or SEQ ID NO. 8. In specific embodiments, the small interfering nucleic acid capable of inhibiting expression of SSX comprises a nucleic acid sequence consisting of SEQ ID NO. 12 or SEQ ID NO. 22. In one embodiment, the small interfering nucleic acid capable of inhibiting expression of GAGE is a duplex having a sense strand consisting of SEQ ID NO. 1 and an antisense strand consisting of SEQ ID NO. 2. In one embodiment, the small interfering nucleic acid capable of inhibiting expression of GAGE is a duplex having a sense strand consisting of SEQ ID NO. 3 and an antisense strand consisting of SEQ ID NO. 4. In one embodiment, the small interfering nucleic acid capable of inhibiting expression of XAGE is a duplex having a sense strand consisting of SEQ ID NO. 5 and an antisense strand consisting of SEQ ID NO. 6. In one embodiment, wherein the small interfering nucleic acid capable of inhibiting expression of XAGE is a duplex having a sense strand consisting of SEQ ID NO. 7 and an antisense strand consisting of SEQ ID NO. 8. In one embodiment, the small interfering nucleic acid capable of inhibiting expression of SSX is a duplex having a sense strand consisting of SEQ ID NO. 11 and an antisense strand consisting of SEQ ID NO. 12. In one embodiment, the small interfering nucleic acid capable of inhibiting expression of SSX is a duplex having a sense strand consisting of SEQ ID NO. 21 and an antisense strand consisting of SEQ ID NO. 22. In some embodiments, the small interfering nucleic acid capable of inhibiting expression of GAGE is a 27-mer siRNA or a small hairpin RNA. In some embodiments, the small interfering nucleic acid capable of inhibiting expression of XAGE is a 27-mer siRNA or a small hairpin RNA. In some embodiments, the small interfering nucleic acid capable of inhibiting expression of SSX is a 27-mer siRNA or a small hairpin RNA.

In some embodiments of the foregoing methods, the cancer cell is in vitro.

In some embodiments of the foregoing methods, the cancer cell is in a subject in need of a treatment effective to inhibit viability, invasion, colony formation and/or migration of the cancer cell.

According to other aspects of the invention, methods for treating an individual having, or suspected of having cancer, are provided. In some embodiments, the methods involve administering to the individual an effective amount of a molecule capable of inhibiting expression of XAGE, a molecule capable of inhibiting expression of GAGE, and/or a molecule capable of inhibiting expression of SSX. In some embodiments, the molecule capable of inhibiting expression of XAGE is or encodes a small interfering nucleic acid capable of inhibiting expression of XAGE, the molecule capable of inhibiting expression of GAGE is or encodes a small interfering nucleic acid capable of inhibiting expression of GAGE, and/or the molecule capable of inhibiting expression of SSX is or encodes a small interfering nucleic acid capable of inhibiting expression of SSX. In some embodiments, the small interfering nucleic acid capable of inhibiting expression of GAGE comprises a nucleic acid sequence consisting of SEQ ID NO. 2 or SEQ ID NO. 4. In some embodiments, the small interfering nucleic acid capable of inhibiting expression of XAGE comprises a nucleic acid sequence consisting of SEQ ID NO. 6 or SEQ ID NO. 8. In some embodiments, the small interfering nucleic acid capable of inhibiting expression of SSX comprises a nucleic acid sequence consisting of SEQ ID NO. 12 or SEQ ID NO. 22. In some embodiments, the small interfering nucleic acid capable of inhibiting expression of GAGE is a duplex having a sense strand consisting of SEQ ID NO. 1 and an antisense strand consisting of SEQ ID NO. 2. In some embodiments, the small interfering nucleic acid capable of inhibiting expression of GAGE is a duplex having a sense strand consisting of SEQ ID NO. 3 and an antisense strand consisting of SEQ ID NO. 4. In some embodiments, the small interfering nucleic acid capable of inhibiting expression of XAGE is a duplex having a sense strand consisting of SEQ ID NO. 5 and an antisense strand consisting of SEQ ID NO. 6. In some embodiments, the small interfering nucleic acid capable of inhibiting expression of XAGE is a duplex having a sense strand consisting of SEQ ID NO. 7 and an antisense strand consisting of SEQ ID NO. 8. In some embodiments, the small interfering nucleic acid capable of inhibiting expression of SSX is a duplex having a sense strand consisting of SEQ ID NO. 11 and an antisense strand consisting of SEQ ID NO. 12. In some embodiments, the small interfering nucleic acid capable of inhibiting expression of SSX is a duplex having a sense strand consisting of SEQ ID NO. 21 and an antisense strand consisting of SEQ ID NO. 22. In some embodiments, the small interfering nucleic acid capable of inhibiting expression of GAGE is a 27-mer siRNA or a small hairpin RNA. In some embodiments, the small interfering nucleic acid capable of inhibiting expression of XAGE is a 27-mer siRNA or a small hairpin RNA. In some embodiments, the small interfering nucleic acid capable of inhibiting expression of SSX is a 27-mer siRNA or a small hairpin RNA.

In some embodiments, the individual has cancer.

In some embodiments, the methods further comprise determining if one or more cancer-testis antigens are expressed in the cancer, optionally wherein the determining is performed prior to administering the molecule(s). In certain embodiments, the one or more cancer-testis antigens is XAGE, GAGE, and/or SSX. In other embodiments, the determining comprises obtaining a sample of the cancer from the individual. In some embodiments, the molecule capable of inhibiting expression of XAGE, the molecule capable of inhibiting expression of GAGE, and/or the molecule capable of inhibiting expression of SSX is combined with a pharmaceutically acceptable carrier.

In some embodiments of the foregoing methods, the cancer cell is a prostate cancer cell.

In some embodiments of the foregoing methods, the cancer cell is a skin cancer cell. In certain embodiments, the skin cancer cell is a melanoma cell.

In some embodiments of the foregoing methods, the cancer cell is a breast cancer cell.

In some embodiments of the foregoing methods, the cancer cell is a lung cancer cell.

### BRIEF DESCRIPTION OF DRAWINGS

FIG. 1 depicts expression of selected CT antigens in normal tissues. An agarose gel shows RT-PCR products of MAGEA1, GAGE, SSX4, CTAG1B, MAGEC1, MAGEC2, XAGE1 and the endogenous control ACTB that were generated by RT-PCR in a panel of 22 normal tissues.

FIG. 2 depicts expression of selected CT antigens in cancer cell lines. An agarose gel shows RT-PCR products of MAGEA1, GAGE, SSX4, CTAG1B, MAGEC1, MAGEC2, XAGE1 and the endogenous control ACTB that were generated by RT-PCR in a panel of 32 cancer cell lines from different origins and testis as a positive control.

FIG. 3 depicts the degree and specificity of gene knock down determined by real-time RT-PCR. SK-MEL-37 cells were transfected with the siRNAs indicated in the first column. Forty-eight hours after transfection cells were harvest for RNA purification and cDNA preparation. Real time PCR was undertaken with the primers and probe sets indicated in the first row and TFRC as endogenous control. Relative quantification of gene expression (relative amount of target RNA) was determined using the equation 2^{-ΔΔCT} using the sample transfected with scrambled siRNA as calibrator.

FIG. 4 depicts the kinetics of siRNA-mediated CT-X knockdown. In FIG. 4A, SK-MEL-37 cells were transfected with 10 nM of siRNA XAGE#2 and cells were harvested for Real-time PCR 3, 6, 12, 18, 24, 48, 96 and every 24 h after that until 240 h. In FIG. 4B, SK-MEL-31 cells were separately transfected with 10 nM of siRNA XAGE#9 and GAGE#9. Cells were harvested for Real-time PCR 48 h after transfection and every 24 h after that until 240 h. In both FIGS. 4A and 4B, relative quantification of gene expression (relative amount of target RNA) was determined using the equation 2^{-ΔΔCT} using the sample transfected with scrambled siRNA as calibrator.

FIG. 5 depicts efficiency of siRNA-mediated CT-X knockdown. Western blot analysis was used to examine the effect of the specific siRNAs on CT-X expression at the protein level, in the cases where antibodies are available (MAGEA, GAGE, SSX, NY-ESO-1, MAGEC1 and MAGEC2). Protein expression was significantly reduced 72 hours after siRNA treatment in SK-MEL-37 cells. Reduction of protein levels to almost complete depletion was present 72 hours after transfection with all six siRNAs.

FIG. 6A depicts an HMGA2 siRNA duplex designed using the algorithm available at Integrated DNA Technologies website (Scitools/Applications/RNAi/RNAi.aspx). This duplex failed to cause knock down of HMGA2 expression. FIG. 6B depicts three prostate cancer cell lines that were independently transfected with HMGA2 siRNA and MAGEA (PC3) or XAGE (22RV1 and DU145) siRNAs. Relative quantification of gene expression was determined using the equation 2^{-ΔΔCT} using the sample transfected with scrambled siRNA as calibrator. While efficient knock down was achieved after transfection with the siRNAs specific to the CT antigens, HMGA2 siRNA failed to knock down HMGA2 in all three cell lines.

FIG. 7 depicts that siRNA duplexes specific to SSX inhibit colony formation in soft agar colony and clonogenic survival of the SK-MEL-37 cell line. In FIG. 7A, at 24 h after transfection with each siRNA, cells were trypsinized, counted and 5,000 cells were seeded in triplicate in plate containing 1% base agar and 0.6% top agar in 6-well plates and allowed to form colonies for 10 days. The number of colonies with 30 cells or larger than 1 mm in diameter in each well was counted. Significantly reduced growth in soft agar in the cells transfected with SSX#12 and SSX#19 was observed as compared to anchorage-independent growth after transfection with non-targeting siRNA. In FIG. 7B, At 24 h after transfection with each siRNA, cells were trypsinized, counted and 1,000 cells were seeded in triplicate in triplicates in 6-well plates and allowed to form colonies for 2 weeks. The colonies were fixed with 10% formalin and stained with 0.2% crystal violet and the number of colonies with 30 cells or larger than 1 mm in diameter in each well was counted. Significantly reduced colony number was observed in the cells transfected with SSX#12 and SSX#19 as compared to anchorage-independent growth after transfection with non-targeting siRNA. All experiments were repeated at least three times and representative data are presented. The knock down levels of SSX in these experiments were confirmed by real-time PCR. Bars, SD. *, P<0.05 relative to non-targeting siRNA (EGFP).

FIG. 8 depicts siRNA duplexes that are specific to XAGE1 inhibit colony formation in soft agar colony and clonogenic survival of SK-MEL-37 cell line. In FIG. 8A, at 24 h after transfection with each siRNA, cells were trypsinized, counted and 5,000 cells were seeded in triplicate in plate containing 1% base agar and 0.6% top agar in 6-well plates and allowed to form colonies for 10 days. The number of colonies with 30 cells or larger than 1 mm in diameter in each well was counted. Significantly reduced growth in soft agar in the cells transfected with XAGE1#2 and XAGE1#9 was observed as compared to anchorage-independent growth after transfection with non-targeting siRNA. In FIG. 8B, at 24 h after transfection with each siRNA, cells were trypsinized, counted and 1,000 cells were seeded in triplicate in triplicates in 6-well plates and allowed to form colonies for 2 weeks. The colonies were fixed with 10% formalin and stained with 0.2% crystal violet and the number of colonies with 30 cells or larger than 1 mm in diameter in each well was counted. Significantly reduced colony number was observed in the cells transfected with XAGE1#2 and XAGE1#9 as compared to anchorage-independent growth after transfection with non-targeting siRNA. All experiments were repeated at least three times and representative data are presented. The knock down levels of SSX in these experiments were confirmed by real-time PCR. Bars, SD. *, P<0.05 relative to non-targeting siRNA (EGFP). FIG. 9 depicts that depletion of GAGE in the melanoma cell lines SK-MEL-37 and SK-MEL-119 results in reduced migration and invasion. In FIGS. 9A and 9B, SK-MEL-37 and SK-MEL-119 cells were treated with nontargeting siRNA or GAGE-specific siRNAs (GAGE#9 and #15). Forty-eight hours later, cells were starved for one hour, seeded onto Boyden chambers and allowed to migrate toward 10% serum for 18 h. After staining with crystal violet, migrating cells were counted under the microscope. In FIGS. 9C and 9D, SK-MEL-37 and SK-MEL-119 cells were treated with nontargeting siRNA or GAGE-specific siRNAs (GAGE#9 and #15). Forty-eight hours later, cells were starved for one hour, seeded onto Matrigel-coated Boyden chambers and allowed to migrate toward 10% serum for 18 h. After staining with crystal violet, cells that invaded the Matrigel layer were counted under the microscope. All experiments were repeated at least three times and representative data are presented. The knock down levels of GAGE in these experiments were confirmed by real-time PCR. Bars, SD. *, P<0.05 relative to non-targeting siRNA (Scrambled siRNA).

FIG. 10 depicts that depletion of XAGE1 in the melanoma cell lines SK-MEL-37, SK-MEL-119, SK-MEL-31 results in reduced migration while the XAGE1 negative cell line SK-MEL-124 is not affected. In FIG. 10A, 10B, and 10C: SK-MEL-37, SK-MEL-119 and SK-MEL-31 cells were treated with nontargeting siRNA or XAGE1-specific siRNAs (XAGE#2 and #9). Forty-eight hours later, cells were starved for one hour, seeded onto Boyden chambers and allowed to migrate toward 10% serum for 18 h. After staining with crystal violet, migrating cells were counted under the microscope. In FIG. 10D, XAGE1 negative SK-MEL-124 cells were treated with nontargeting siRNA or XAGE-specific siRNAs (XAGE#2 and #9). Forty-eight hours later, cells were starved for one hour, seeded onto Boyden chambers and allowed to migrate toward 10% serum for 18 h. After staining with crystal violet, cells that migrated were counted under the microscope. All experiments were repeated at least three times and representative data are presented. The knock down levels of XAGE1 in these experiments were confirmed by real-time PCR and regular RT-PCR with XAGE1 isoform-specific primers and the agarose gels with the amplification products are shown at the bottom of each graph. Bars, SD. *, P<0.05 relative to non-targeting siRNA (Scrambled siRNA).

FIG. 11 depicts depletion of XAGE1 in the melanoma cell lines SK-MEL-37 and SK-MEL-119 results in reduced invasion. SK-MEL-37 (11A) and SK-MEL-119 (11B) cells were treated with nontargeting siRNA or XAGE1-specific siRNAs (GAGE#2 and #9). Forty-eight hours later, cells were starved for one hour, seeded onto Matrigel-coated Boyden chambers and allowed to migrate toward 10% serum for 18 h. After staining with crystal violet, cells that invaded the Matrigel layer were counted under the microscope. All experiments were repeated at least three times and representative data are presented. The knock down levels of XAGE1 in these experiments were confirmed by real-time PCR. Bars, SD. *, P<0.05 relative to non-targeting siRNA (Scrambled siRNA).

FIG. 12 depicts that depletion of XAGE1 results in reduced migration and viability in prostate cancer and NSCLC cell lines. NSCLC cell line SK-LC-5 (12A) and prostate cancer cell line DU145 (12B) were treated with nontargeting siRNA or XAGE1-specific siRNAs (XAGE#2 and #9). Forty-eight hours later, cells were starved for one hour, seeded onto Boyden chambers and allowed to migrate toward 10% serum for 18 h. After staining with crystal violet, migrating cells were counted under the microscope. At 24 h after transfection with each siRNA, SK-LC-5 cells (12C) and 22RV1 (12D) were trypsinized, counted and 1,000 cells were seeded in triplicate in triplicates in 6-well plates and allowed to form colonies for 2 weeks. The colonies were fixed with 10% formalin and stained with 0.2% crystal violet and the number of colonies with 30 cells or larger than 1 mm in diameter in each well was counted. Significantly reduced colony number was observed in the cells transfected with XAGE1#2 and XAGE1#9 in SK-LC5 and XAGE1#2 in 22RV1 as compared to cells transfected with non-targeting siRNA. All experiments were repeated at least three times and representative data are presented. The knock down levels of XAGE1 in these experiments were confirmed by real-time PCR. Bars, SD. *, P<0.05 relative to non-targeting siRNA (Scrambled siRNA).

### DETAILED DESCRIPTION OF THE INVENTION

The T cell epitope cloning technique developed by Boon et al in 1991 led to the discovery of the human tumor antigens MAGE1, BAGE and GAGE1 (Van den Eynde, B. et al, J Exp Med., 1991 Jun. 1; 173(6):1373-84; Van den Eynde, B. et al, J Exp Med., 1995 Sep. 1; 182(3):689-98). The mRNA transcripts encoding these gene products were present exclusively in normal testis tissues. These genes and several others were also found using serological expression cloning (SEREX) to identify tumor antigens having high immunogenicity (Sahin U. et al, Proc Natl Acad Sci U S A, 1995 Dec. 5; 92(25):11810-3). Due to the fact that these genes are primarily expressed in spermatogonia and in normal testis, showing restricted expression in normal tissues, they were catalogued as Cancer-testis (CT) antigens. Since then, forty-four CT antigen genes or gene families have been identified by immunological or genetic approaches (Scanlan, M. J. et al, Cancer Immun., 2004 Jan. 23; 4:1). Some thoroughly studied CT antigens are MAGE, BAGE and LAGE/NY-ESO-1 (Jungbluth, A. A. et al, Int J Cancer., 2001 Jun. 15; 92(6):856-60; Scanlan, M. J. et al, Immunol Rev., 2002 October; 188:22-32; Gnjatic S. et al, Adv Cancer Res., 2006; 95:1-30). Several CT antigens have been shown to elicit spontaneous humoral and cellular immune responses in cancer patients simultaneously (Jäger, E. et al, J Exp Med., 1998 Jan. 19; 187(2):265-70; Ayyoub, M. et al, J Immunol. 2002 Feb. 15; 168(4):1717-22). Initial expression studies of CT antigens were mostly done at the level of mRNA expression by RT-PCR. Studies of the expression of CT antigens at the protein level provide important information regarding their distribution in tumor samples, as shown in studies of the MAGE, NY-ESO-1 and SSX families (Juretic, A. et al, Lancet Oncol., 2003 February; 4(2):104-9).

The invention disclosed herein relates to the development and use of two specific siRNA molecules of 27 nucleotides in length (“27 mers”) that inhibit the expression and function of two proteins that are members of the Cancer-testis antigens (CT) family. Both of the 27 mer siRNAs provide better knock-down of the genes than classical 21 mer siRNAs. The siRNAs are used to deplete XAGE1 (variants 1-3) and GAGE (variants 1,2,3,4,5,6,7B and 8) in cancer cell lines. The invention further relates to the discovery that inhibition of the expression of the XAGE and GAGE genes causes reduction in migration, invasion, and viability specifically in cancer cells. Thus, some embodiments of the invention are cancer cell specific therapeutic strategies for inhibiting metastasis and/or viability of malignant tumors.

The XAGE-1 gene, referred to herein also as XAGE, was originally identified as a PAGE/GAGE-related gene on the X chromosome by EST analysis (Brinkmann U. et al, Cancer Res., 1999 Apr. 1; 59(7):1445-8). The expression profile of XAGE-1 suggested that it has the characteristics of a CT antigen (Boon, T. et al, Curr Opin Immunol., 1997 Oct. 1; 9(5):681-3; Scanlan, M. J. et al, Immunol Rev., 2002 October; 188:22-32; Liu, X. F. et al, Cancer

Res., 2000 Sep. 1; 60(17):4752-5). Transcription of the XAGE-1 gene is regulated by methylation of the CpG island in the promoter, and 4 alternative RNA splicing variants, XAGE-1a, b, c and d, have been identified (Zendman, A. J. et al, Int J Cancer., 2002 May 20; 99(3):361-9; Lim, J. H. et al, Int J Cancer., 2005 Aug. 20; 116(2):200-6). By serological analysis of antigens by recombinant expression cloning (SEREX), Wang et al identified XAGE-1b as a dominant antigen recognized by serum from a lung adenocarcinoma patient using an autologous tumor cell line and showed that XAGE-1b is immunogenic in patients with lung adenocarcinoma (Wang, T. et al, Oncogene, 2001 Nov. 22; 20(53):7699-709). Overlapping XAGE-1 transcripts encoding a cancer testis antigen have been found expressed in lung, breast, and other types of cancers (Egland, K. A. et al, Mol Cancer Ther., 2002 May; 1(7):441-50). Antibody response against XAGE-1 was found in patients with prostate cancer (Koizumi, F., et al, Microbiol Immunol., 2005; 49(5):471-6), non-small cell lung cancer (Nakagawa, K. et al, Clin Cancer Res., 2005 Aug. 1; 11(15):5496-503) and melanoma metastasis (Zendman, A. J. et al, Int J Cancer., 2002 Jan. 10; 97(2):195-204; Zendman, A. J. et al, Int J Cancer., 2002 May 20; 99(3):361-9). Several variants of XAGE-1b were found to be predominantly expressed in testis and tumors (Sato, S. et al, Cancer Immun., 2007 Mar. 5; 7:5).

GAGE1 and GAGE2 were first described as antigens recognized by autologous cytolytic T lymphocytes on a human melanoma by Boon et al (Van den Eynde, B. et al, J Exp Med., 1995 Sep. 1; 182(3):689-98). As GAGE1 and 2, new members of this family GAGE1,2,3,4,5,6,7B and 8 were found to be absent from normal tissues but testis and expressed in a variety of cancer tissues as melanomas (24%), sarcomas (25%), non-small cell lung cancers (19%), head and neck tumors (19%), and bladder tumors (12%) (De Backer, O. et al, Cancer Res., 1999 Jul. 1; 59(13):3157-65). GAGE proteins have been proposed to be a potential target for specific immunotherapy and diagnostic markers by several labs for several tumor types. Publications describing expression of GAGE in melanoma tissues and cell lines (Bazhin, A. V. et al, Cancer Lett., 2007 Jun. 28; 251(2):258-67. Epub 2006 Dec. 27), poor survival in melanoma patients (Cheung, I. Y. et al, Clin Cancer Res., 1999 August; 5(8):2042-7), expression in melanoma metastasis (Dalerba, P. et al, Int J Cancer., 1998 Jul. 17; 77(2):200-4), prostate cell line LNCaP (Chen, M. E. et al, J Biol Chem., 1998 Jul. 10; 273(28):17618-25) also in metastatic neuroblastoma (Cheung, I. Y. et al, Med Pediatr Oncol., 2000 December;35(6):632-4) and uterine cervical carcinoma (Chang, H. K. et al, Gynecol Oncol., 2005 May; 97(2):342-7; Brinkmann, U. et al, Cancer Res., 1999 Apr. 1; 59(7):1445-8).

Cancer is a disease characterized by uncontrolled cell proliferation and other malignant cellular properties. Cancer cells can arise from a number of genetic and epigenetic perturbations that cause defects in mechanisms controlling cell migration, invasion, proliferation, survival, differentiation, and growth that lead to tumor formation and/or metastasis. As used herein, the term cancer includes, but is not limited to, the following types of cancer: breast cancer; biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will be known to one of ordinary skill in the art. In one embodiment the cancer is melanoma. In one embodiment the cancer is prostate cancer. In one embodiment the cancer is lung cancer. In one embodiment the cancer is breast cancer.

Tumors resulting from uncontrolled cell proliferation can be either benign or malignant. Whereas benign tumors remain localized in a primary tumor that remains localized at the site of origin and that is often self limiting in terms of tumor growth, malignant tumors have a tendency for sustained growth and an ability to spread or metastasize to distant locations. Metastasis, as used herein, refers to this spreading of malignant tumor cells and involves a diverse repertoire of malignant properties. These metastatic properties, as used herein, include cell invasion into tissues adjacent to primary tumors, migration through adjacent tissue, entry into the bloodstream or lymphatic system, dissemination through the bloodstream or lymphatic system, exit from the bloodstream or lymphatic system, and implantation at distant sites where new tumors can form. Other metastatic properties include aberrant cell proliferation, growth, survival. Thus, tumor metastasis involves, at least in part, the ability of metastatic cells to adhere to the proteins of the extracellular matrix (ECM), to migrate, and to survive at a distant location. In one embodiment the invention involves inhibition of the expression of the XAGE and GAGE genes to inhibit properties of tumor metastasis including, migration, invasion, and viability, in cancer cells.

As used herein, inhibitors of tumor metastasis are molecules (inhibitor molecules) that affect one or more tumor metastatic properties. For example, tumor metastatic properties that can be affected include cell migration, invasion, proliferation, and viability. As used herein, “inhibition” or “inhibiting” refers to the reduction or suppression of, for example, tumor metastasis or a tumor metastatic property. Inhibition may, or may not, be complete. For example, cell proliferation may, or may not, be decreased to a state of complete arrest for the effect of a molecule to be considered one of inhibition. Moreover, inhibition may include the prevention of the acquisition of metastatic properties, and the reduction of already existing metastatic properties, for example invasion or migration.

In one embodiment, “inhibition” relates to cancer cell viability. “Viability”, as used herein may refer to a cell's capacity for survival, or just survival of a cell. Thus, in some aspects, inhibitors of cell viability are molecules (e.g., small interfering nucleic acids) that make tumor cells more susceptible to death. In other aspects, inhibitors of cell viability are molecules that kill tumor cells. Inhibition may, or may not, be complete. For example, it is not necessary that all tumor cells be killed in a population of tumor cells (e.g., in a tumor) that is targeted by an inhibitor molecule, for the effect of the molecule to be considered one of inhibition of viability.

As used herein, “isolated” nucleic acid refers to a nucleic acid (e.g., DNA, RNA, etc . . . ) that has been removed from its native environment. For example, an RNA (e.g., siRNA) purified (partially or substantially) from a cell is an isolated nucleic acid. As used herein, “isolated” nucleic acid also refers to a nucleic acid that has been synthesized in a non-natural setting. For example, a small-interfering nucleic acid synthesized using an automated nucleic acid synthesizer, examples of which are well known in the art, is an isolated nucleic acid.

In particular, the invention features inhibitor molecules that are small interfering nucleic acids (siNA), which include, small interfering RNA (siRNA), double-stranded RNA (dsRNA), micro-RNA (miRNA), and short hairpin RNA (shRNA) molecules, and that are used to inhibit the expression of target genes. The siNAs of the present invention, for example siRNAs, typically regulate gene expression via target RNA transcript cleavage/degradation or translational repression of the target messenger RNA (mRNA). In one embodiment siRNAs are exogenously delivered to a cell.

In some embodiments, inhibitor molecules comprising the following siRNA sequences are featured (RIBONUCLEOTIDES are in upper case and deoxyribonucleotides are underlined in lower case), but other combinations of ribonucleotides and deoxyribonucleotides are also possible as will be known to one of ordinary skill in the art:

### Duplex name ACC NM_{—}001468_{—}15—GAGE#15

| Sense Sequence (5′-3′) (Position:249) | |
|---|---|
| GAACCAGCAACUCAACGUCAGGAtc | (SEQ ID NO: 1) |
| Antisense Sequence (5′-3′) (Position:273) | |
| GAUCCUGACGUUGAGUUGCUGGUUCCC | (SEQ ID NO: 2) |

Asymmetrical End Stability Difference: −0.41
Duplex identity:100% with the following mRNA targets:

NM_{—}001098411.3 Homo sapiens G antigen 2B (GAGE2B)

NM_{—}001127212.1 Homo sapiens G antigen 2A (GAGE2A)

NM_{—}001127200.1 Homo sapiens G antigen 2E (GAGE2E)

NM_{—}001098413.2 Homo sapiens G antigen 10 (GAGE10)

NM_{—}001098405.1 Homo sapiens G antigen 12F (GAGE12F)

NM_{—}001098407.1 Homo sapiens G antigen 2D (GAGE2D)

NM_{—}001098409.1 Homo sapiens G antigen 12G (GAGE12G)

NM_{—}001098406.1 Homo sapiens G antigen 12J (GAGE12J)

NM_{—}001472.2 Homo sapiens G antigen 2C (GAGE2C)

NM_{—}001468.3 Homo sapiens G antigen 1 (GAGE!)

NM_{—}001040663.1 Homo sapiens G antigen 1 (GAGE1)

NM_{—}021123.2 Homo sapiens G antigen 7 (GAGE7)

NM_{—}001477.1 Homo sapiens G antigen 12I (GAGE12I)

NM_{—}012196.1 Homo sapiens G antigen 8 (GAGES)

NM_{—}001476.1 Homo sapiens G antigen 6 (GAGE6)

NM_{—}001475.1 Homo sapiens G antigen 5 (GAGES)

NM_{—}001474.1 Homo sapiens G antigen 4 (GAGE4)

### Duplex name ACC NM_{—}001468_{—}9—GAGE#9

Sense Sequence (5′-3′) (Position: 209)

| | |
|---|---|
| GUUCAGUGAUGAAGUGGAACCAGca | (SEQ ID NO: 3) |

AntiSense Sequence (5′-3′) (Position: 233)

| | |
|---|---|
| UGCUGGUUCCACUUCAUCACUGAACUG | (SEQ ID NO: 4) |

Asymmetrical End Stability Difference: −1.02
Duplex identity:100% with the following mRNA targets:

NM_{—}001098411.3 Homo sapiens G antigen 2B (GAGE2B)

NM_{—}001127212.1 Homo sapiens G antigen 2A (GAGE2A)

NM_{—}001127200.1 Homo sapiens G antigen 2E (GAGE2E)

NM_{—}001127199.1 Homo sapiens G antigen 12D (GAGE12D)

XM_{—}001713660.1 PREDICTED: Homo sapiens G antigen 12D (GAGE12D)

NM_{—}001098413.2 Homo sapiens G antigen 10 (GAGE10)

NM_{—}001098418.1 Homo sapiens G antigen 12E (GAGE12E)

NM_{—}001098408.1 Homo sapiens G antigen 12C (GAGE12C)

NM_{—}001098410.1 Homo sapiens G antigen 12H (GAGE12H)

NM_{—}001098405.1 Homo sapiens G antigen 12F (GAGE12F)

NM_{—}001098407.1 Homo sapiens G antigen 2D (GAGE2D)

NM_{—}001098409.1 Homo sapiens G antigen 12G (GAGE12G)

NM_{—}001098406.1 Homo sapiens G antigen 12J (GAGE12J)

NM_{—}001085441.1 Homo sapiens G antigen 12D (GAGE12D)

NM_{—}001127345.1 Homo sapiens G antigen 12B (GAGE12B)

NM_{—}001472.2 Homo sapiens G antigen 2C (GAGE2C)

NM_{—}001468.3 Homo sapiens G antigen 1 (GAGE1)

NM_{—}001040663.1 Homo sapiens G antigen 1 (GAGE1)

NM_{—}021123.2 Homo sapiens G antigen 7 (GAGE7)

NM_{—}001477.1 Homo sapiens G antigen 12I (GAGE12I)

NM_{—}012196.1 Homo sapiens G antigen 8 (GAGES)

NM_{—}001476.1 Homo sapiens G antigen 6 (GAGE6)

NM_{—}001475.1 Homo sapiens G antigen 5 (GAGES)

NM_{—}001474.1 Homo sapiens G antigen 4 (GAGE4)

### Duplex name: ACC NM_{—}133430_{—}2—XAGE1#2

| Sense Sequence (5′-3′) (Position: 186) | |
|---|---|
| GACAGAAGAAGAUCAGGAUACAGct | (SEQ ID NO: 5) |
| Antisense Sequence (5′-3′) (Position:210) | |
| AGCUGUAUCCUGAUCUUCUUCUGUCUG | (SEQ ID NO: 6) |

Asymmetrical End Stability Difference: −0.01
Duplex identity: 100% with the following mRNA targets:

NM_{—}133431.2 Homo sapiens X antigen family, member 1D (XAGE1D), transcript variant

NM_{—}001097596.1 Homo sapiens X antigen family, member 1B (XAGE1B), transcript variant

NM_{—}001097594.1 Homo sapiens X antigen family, member 1B (XAGE1B), transcript variant

NM_{—}001097591.1 Homo sapiens X antigen family, member 1A (XAGE1A), transcript variant

NM_{—}001097593.1 Homo sapiens X antigen family, member 1A (XAGE1A), transcript variant

NM_{—}001097605.1 Homo sapiens X antigen family, member 1E (XAGE1E), transcript variant

NM_{—}001097603.1 Homo sapiens X antigen family, member 1E (XAGE1E), transcript variant

NM_{—}001097602.1 Homo sapiens X antigen family, member 1C (XAGE1C), transcript variant

NM_{—}001097595.1 Homo sapiens X antigen family, member 1B (XAGE1B), transcript variant

NM_{—}001097597.1 Homo sapiens X antigen family, member 1C (XAGE1C), transcript variant

NM_{—}001097604.1 Homo sapiens X antigen family, member 1E (XAGE1E), transcript variant

NM_{—}001097598.1 Homo sapiens X antigen family, member 1C (XAGE1C), transcript variant

NM_{—}001097592.1 Homo sapiens X antigen family, member 1A (XAGE1A), transcript variant

NM_{—}020411.1 Homo sapiens X antigen family, member 1D (XAGE1D), transcript variant

NM_{—}133430.1 Homo sapiens X antigen family, member 1D (XAGE1D), transcript variant

### Duplex name: ACC NM_{—}133430_{—}9—XAGE1#9

Sense Sequence (5′-3′) (Position: 395)

| | |
|---|---|
| AAGCUGAAACAACGCAAGCUGGUtt | (SEQ ID NO: 7) |

AntiSense Sequence (5′-3′) (Position: 419)

| | |
|---|---|
| AAACCAGCUUGCGUUGUUUCAGCUUGU | (SEQ ID NO: 8) |

Asymmetrical End Stability Difference: −0.03
Duplex identity: 100% with the following mRNA targets:

NM_{—}133431.2 Homo sapiens X antigen family, member 1D (XAGE1D), transcript variant 2

NM_{—}001097596.1 Homo sapiens X antigen family, member 1B (XAGE1B), transcript variant 3

NM_{—}001097594.1 Homo sapiens X antigen family, member 1B (XAGE1B), transcript variant 2

NM_{—}001097591.1 Homo sapiens X antigen family, member lA (XAGE1A), transcript variant 1

NM_{—}001097593.1 Homo sapiens X antigen family, member lA (XAGE1A), transcript variant 3

NM_{—}001097605.1 Homo sapiens X antigen family, member lE (XAGE1E), transcript variant 3

NM_{—}001097603.1 Homo sapiens X antigen family, member lE (XAGE1E), transcript variant 1

NM_{—}001097602.1 Homo sapiens X antigen family, member 1C (XAGE1C), transcript variant 1

NM_{—}001097595.1 Homo sapiens X antigen family, member 1B (XAGE1B), transcript variant 1

NM_{—}001097597.1 Homo sapiens X antigen family, member 1C (XAGE1C), transcript variant 2

NM_{—}001097604.1 Homo sapiens X antigen family, member lE (XAGE1E), transcript variant 2

NM_{—}001097598.1 Homo sapiens X antigen family, member 1C (XAGE1C), transcript variant 3

NM_{—}001097592.1 Homo sapiens X antigen family, member lA (XAGE1A), transcript variant 2

XM_{—}001143525.1 PREDICTED: Pan troglodytes G antigen, family D, 2, transcript variant 1

NM_{—}020411.1 Homo sapiens X antigen family, member 1D (XAGE1D), transcript variant 1

NM_{—}133430.1 Homo sapiens X antigen family, member 1D (XAGE1D), transcript variant 3

### Duplex name: ACC NM_{—}005462_{—}19—MAGEC1

| Sense Sequence (Position: 2437) | |
|---|---|
| GGAGGACUCCCUCUCUCCUCUCCac | (SEQ ID NO: 9) |
| Antisense Sequence (Position: 2461) | |
| GUGGAGAGGAGAGAGGGAGUCCUCCCA | (SEQ ID NO: 10) |

Asymmetrical End Stability Difference: 1.18
Duplex identity: 100% with the following mRNA target:
NM_{—}005462.3 Homo sapiens melanoma antigen family C, 1 (MAGEC1)

### Duplex name: ACC NM_{—}005636_{—}12—SSX4#12

| Sense Sequence (Position: 586) | |
|---|---|
| CAAGGUCACCCUCCCACCUUUCAtg | (SEQ ID NO: 11) |
| Antisense Sequence (Position: 610) | |
| CAUGAAAGGUGGGAGGGUGACCUUGAA | (SEQ ID NO: 12) |

Asymmetrical End Stability Difference: 0.86
Duplex identity: 100% with the following mRNA target:

XM_{—}001725018.1 PREDICTED: Homo sapiens synovial sarcoma, X breakpoint 4 (SSX4)

NM_{—}001040612.1 Homo sapiens synovial sarcoma, X breakpoint 4B (SSX4B), transcript variant 2

NM_{—}001034832.2 Homo sapiens synovial sarcoma, X breakpoint 4B (SSX4B), transcript variant 1

NM_{—}175729.1 Homo sapiens synovial sarcoma, X breakpoint 4 (SSX4), transcript variant 2

NM_{—}005636.3 Homo sapiens synovial sarcoma, X breakpoint 4 (SSX4), transcript variant 1

NM_{—}175698 Homo sapiens synovial sarcoma, X breakpoint 4 (SSX2), transcript variant 2

NM_{—}003147 Homo sapiens synovial sarcoma, X breakpoint 4 (SSX2), transcript variant 1

### Duplex name: ACC NM_{—}005636_{—}19—SSX4#19

| Sense Sequence (5′-3′) (Position: 892 ) | |
|---|---|
| CUUGUGUAUCCAUGCACCUACCUca | (SEQ ID NO: 21) |
| Antisense Sequence (5′-3′) (Position: 916) | |
| UGAGGUAGGUGCAUGGAUACACAAGCC | (SEQ ID NO: 22) |

Asymmetrical End Stability Difference: −2.33
Duplex identity: 100% with the following mRNA targets:

XM_{—}001725018.1 PREDICTED: Homo sapiens synovial sarcoma, X breakpoint 4 (SSX4)

NM_{—}001040612.1 Homo sapiens synovial sarcoma, X breakpoint 4B (SSX4B), transcript variant 2

NM_{—}001034832.2 Homo sapiens synovial sarcoma, X breakpoint 4B (SSX4B), transcript variant 1

NM_{—}173357.2 Homo sapiens synovial sarcoma, X breakpoint 6 (SSX6)

NM_{—}175729.1 Homo sapiens synovial sarcoma, X breakpoint 4 (SSX4), transcript variant 2

NM_{—}005636.3 Homo sapiens synovial sarcoma, X breakpoint 4 (SSX4), transcript variant 1

NM_{—}175698 Homo sapiens synovial sarcoma, X breakpoint 4 (SSX2), transcript variant 2

NM_{—}003147 Homo sapiens synovial sarcoma, X breakpoint 4 (SSX2), transcript variant 1

### Duplex name: ACC NM_{—}001327_{—}7—NY-ESO-1 (CTAG1B)

| Sense sequence (Position:451) | |
|---|---|
| GCUUCUGAAGGAGUUCACUGUGUcc | (SEQ ID NO: 13) |
| Antisense sequence (Position:475) | |
| GGACACAGUGAACUCCUUCAGAAGCAC | (SEQ ID NO: 14) |

Asymmetrical End Stability Difference: 0

### Duplex identity: 100% with the following mRNA targets:

NM_{—}139250.1 Homo sapiens cancer/testis antigen 1A (CTAG1A)

NM_{—}001327.1 Homo sapiens cancer/testis antigen 1B (CTAG1B)

### Duplex name: ACC NM_{—}005362_{—}3 MAGEA

Sense Sequence (5′-3′) (Position: 1051)

| | |
|---|---|
| CCAGCUAUGUGAAAGUCCUGCACca | (SEQ ID NO: 23) |

Antisense Sequence (5′-3′) (Position: 1075)

| | |
|---|---|
| UGGUGCAGGACUUUCACAUAGCUGGUU | (SEQ ID NO: 24) |

Asymmetrical End Stability Difference: 0.94
Duplex identity: 100% with the following mRNA targets:

NM_{—}005362.3 Homo sapiens melanoma antigen family A, 3 (MAGEA3)

NM_{—}005363.2 Homo sapiens melanoma antigen family A, 6 (MAGEA6), transcript variant 1

NM_{—}175868.1 Homo sapiens melanoma antigen family A, 6 (MAGEA6), transcript variant 2

NM_{—}153488.3 Homo sapiens melanoma antigen family A, 2B (MAGEA2B)

NM_{—}175743.1 Homo sapiens melanoma antigen family A, 2 (MAGEA2), transcript variant 3

NM_{—}175742.1 Homo sapiens melanoma antigen family A, 2 (MAGEA2), transcript variant 2

NM_{—} 005361.2 Homo sapiens melanoma antigen family A, 2 (MAGEA2), transcript variant 1

NM_{—}005367.4 Homo sapiens melanoma antigen family A, 12 (MAGEA12)

### Duplex name: ACC NM_{—}016249_{—}3 MAGEC2#3

| Sense Sequence (5′-3′) (Position: 873 ) | |
|---|---|
| AGAUUACUUUCCUGUGAUACUCAag | (SEQ ID NO: 25) |
| Antisense Sequence (5′-3′) (Position:897) | |
| CUUGAGUAUCACAGGAAAGUAAUCUUU | (SEQ ID NO: 26) |

Asymmetrical End Stability Difference: −0.43
Duplex identity: 100% with the following mRNA targets:

NM_{—}016249.2 Homo sapiens melanoma antigen family C, 2 (MAGEC2)

Duplex name: ACC NM_{—}016249_{—}17 MAGEC2#17

| Sense Sequence (5′-3′) (Position:1545 ) | |
|---|---|
| CUCGAGGAACGUAGUGUUCUUUGca | (SEQ ID NO: 27) |
| Antisense Sequence (5′-3′) (Position:1569) | |
| UGCAAAGAACACUACGUUCCUCGAGCC | (SEQ ID NO: 28) |

Asymmetrical End Stability Difference: 1.51
Duplex identity: 100% with the following mRNA targets:

NM_{—}016249.2 Homo sapiens melanoma antigen family C, 2 (MAGEC2)

Examples of the foregoing duplex inhibitors molecules are depicted in the following schematics:

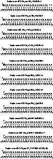

A small interfering nucleic acid (siNA) of the invention can be unmodified or chemically-modified. A siNA of the instant invention can be chemically synthesized, expressed from a vector or enzymatically synthesized. The instant invention also features various chemically-modified synthetic small interfering nucleic acid (siNA) molecules capable of inhibiting gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. For example, in some cases, siRNAs are modified to alter potency, target affinity, the safety profile and/or the stability to render them resistant or partially resistant to intracellular degradation. Modifications, such as phosphorothioates, for example, can be made to siRNAs to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006). siRNAs with amide-linked oligoribonucleosides have been generated that are more resistant to S1 nuclease degradation (Iwase R et al. 2006 Nucleic Acids Symp Ser 50: 175-176). In addition, modification of siRNA at the 2′-sugar position and phosphodiester linkage confers improved serum stability without loss of efficacy (Choung et al., Biochem. Biophys. Res. Commun. 342(3):919-26, 2006). In one study, 2′-deoxy-2′-fluoro-beta-D-arabinonucleic acid (FANA)-containing antisense oligonucleotides compared favourably to phosphorothioate oligonucleotides, 2′-O-methyl-RNA/DNA chimeric oligonucleotides and siRNAs in terms of suppression potency and resistance to degradation (Ferrari N et a. 2006 Ann N Y Acad Sci 1082: 91-102).

In some embodiments an siNA is an shRNA molecule encoded by and expressed from a genomically integrated transgene or a plasmid-based expression vector. Thus, in some embodiments a molecule capable of inhibiting gene expression is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

One embodiment herein contemplates the use of gene therapy to deliver one or more expression vectors, for example viral-based gene therapy, encoding one or more small interfering nucleic acids, capable of inhibiting expression of XAGE and/or a molecule capable of inhibiting expression of GAGE. As used herein, gene therapy is a therapy focused on treating genetic diseases, such as cancer, by the delivery of one or more expression vectors encoding therapeutic gene products, including polypeptides or RNA molecules, to diseased cells. Methods for construction and delivery of expression vectors will be known to one of ordinary skill in the art.

Other molecules that can be used to inhibit gene expression include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9,1996). Multitarget ribozymes (connected or shotgun) have been suggested as a means of improving efficiency of ribozymes for gene inhibition (Ohkawa et al., Nucleic Acids Symp Ser. (29):121-2, 1993).

Triple helix approaches have also been investigated for sequence-specific gene inhibition. Triplex forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for future inhibition at the DNA level (Trauger et al., Chem. Biol. 3(5):369-77, 1996). In addition, inhibition has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). In some cases inhibition strategies have lead to a reduction in RNA levels without a concomitant reduction in proteins, whereas in others, reductions in RNA have been mirrored by reductions in protein.

One aspect of the invention contemplates the treatment of a subject, also referred to as an individual, having or at risk of having cancer. As used herein a subject is a mammalian species, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), zoo animals (e.g., lions, giraffes, etc.), but are not so limited. Preferred subjects are human subjects. The human subject may be a pediatric, adult or a geriatric subject.

As used herein treatment, or treating, includes amelioration, cure or maintenance (i.e., the prevention of relapse) of a disorder. Treatment after a disorder has started aims to reduce, ameliorate or altogether eliminate the disorder, and/or its associated symptoms, to prevent it from becoming worse, or to prevent the disorder from re-occurring once it has been initially eliminated (i.e., to prevent a relapse).

The invention in other embodiments provides a pharmaceutical pack or kit comprising one or more containers filled with one or more of the ingredients of the pharmaceutical compositions of the invention. Associated with such container(s) can be various written materials such as instructions (indicia) for use, or a notice in the form prescribed by a governmental agency regulating the manufacture, use or sale of pharmaceuticals or biological products, which notice reflects approval by the agency of manufacture, use or sale for human administration.

The pharmaceutical compositions of the present invention preferably contain a pharmaceutically acceptable carrier or excipient suitable for rendering the compound or mixture administrable orally as a tablet, capsule or pill, or parenterally, intravenously, intradermally, intramuscularly or subcutaneously, or transdermally. The active ingredients may be admixed or compounded with any conventional, pharmaceutically acceptable carrier or excipient.

As used herein, the term “pharmaceutically acceptable carrier” includes any and all solvents, dispersion media, coatings, antibacterial and antifungal agents, isotonic agents, absorption delaying agents, and the like. The use of such media and agents for pharmaceutically active substances is well known in the art. Except insofar as any conventional media or agent is incompatible with the compositions of this invention, its use in the therapeutic formulation is contemplated. Supplementary active ingredients can also be incorporated into the pharmaceutical formulations.

It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 4th ed. (1970), the disclosure of which is incorporated herein by reference. Those skilled in the art, having been exposed to the principles of the invention, will experience no difficulty in determining suitable and appropriate vehicles, excipients and carriers or in compounding the active ingredients therewith to form the pharmaceutical compositions of the invention.

An effective amount, also referred to as a therapeutically effective amount, of a gene expression inhibitor molecule (for example, a siNA molecule capable of inhibiting expression of XAGE or a molecule capable of inhibiting expression of GAGE) is an amount sufficient to ameliorate at least one adverse effect associated with expression of the gene in a cell (for example, a cancer cell) or in an individual in need of such gene inhibition (for example, an individual having cancer). The therapeutically effective amount the gene expression inhibitor molecule (active agent) to be included in pharmaceutical compositions depends, in each case, upon several factors, e.g., the type, size and condition of the patient to be treated, the intended mode of administration, the capacity of the patient to incorporate the intended dosage form, etc. Generally, an amount of active agent is included in each dosage form to provide from about 0.1 to about 250 mg/kg, and preferably from about 0.1 to about 100 mg/kg. One of ordinary skill in the art would be able to determine empirically an appropriate therapeutically effective amount.

While it is possible for the agents to be administered as the raw substances, it is preferable, in view of their potency, to present them as a pharmaceutical formulation. The formulations of the present invention for human use comprise the agent, together with one or more acceptable carriers therefor and optionally other therapeutic ingredients. The carrier(s) must be “acceptable” in the sense of being compatible with the other ingredients of the formulation and not deleterious to the recipient thereof or deleterious to the inhibitory function of the active agent. Desirably, the formulations should not include oxidizing agents and other substances with which the agents are known to be incompatible. The formulations may conveniently be presented in unit dosage form and may be prepared by any of the methods well known in the art of pharmacy. All methods include the step of bringing into association the agent with the carrier, which constitutes one or more accessory ingredients. In general, the formulations are prepared by uniformly and intimately bringing into association the agent with the carrier(s) and then, if necessary, dividing the product into unit dosages thereof.

Formulations suitable for parenteral administration conveniently comprise sterile aqueous preparations of the agents, which are preferably isotonic with the blood of the recipient. Suitable such carrier solutions include phosphate buffered saline, saline, water, lactated ringers or dextrose (5% in water). Such formulations may be conveniently prepared by admixing the agent with water to produce a solution or suspension, which is filled into a sterile container and sealed against bacterial contamination. Preferably, sterile materials are used under aseptic manufacturing conditions to avoid the need for terminal sterilization.

Such formulations may optionally contain one or more additional ingredients among which may be mentioned preservatives, such as methyl hydroxybenzoate, chlorocresol, metacresol, phenol and benzalkonium chloride. Such materials are of special value when the formulations are presented in multidose containers.

Buffers may also be included to provide a suitable pH value for the formulation. Suitable such materials include sodium phosphate and acetate. Sodium chloride or glycerin may be used to render a formulation isotonic with the blood. If desired, the formulation may be filled into the containers under an inert atmosphere such as nitrogen or may contain an anti-oxidant, and are conveniently presented in unit dose or multi-dose form, for example, in a sealed ampoule.

The practice of the present invention employs, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies : a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### EXAMPLES

### Example 1

### Material and Methods

27mer siRNA Oligonucleotide Design—Dicer Substrate RNAs:

Dicer-Substrate RNAs are chemically synthesized 27-mer RNA duplexes that are optimized for Dicer processing and show increased potency when compared with 21-mer duplexes [1, 2]. The duplexes were chosen by a rational design algorithm that integrates both traditional 21-mer siRNA design rules as well as new 27-mer design criteria available at IDT's website (idtdna.com/Scitools/Applications/RNAi/RNAi.aspx). The approximately 20 options identified by the algorithm in each case were optimized at several levels. We first level aimed to exclude off-target complementarity. This was undertaken with the BLAST tool at NCBI's website with an adjustment for analyzing short sequences (ncbi.nlm.nih.gov/BLAST/). Sequences were excluded if total or partial complementarity with other genes was noted. Further selection was based on published criteria for selection of active siRNA[3, 4] that included:
Avoiding non-coding region and sequence following the start codon (75-100 bp) to prevent the targeting of regions of mRNA occupied by translational or regulatory proteins or regions that are potentially polymorphic.
G-C content from 30 to 70%
Avoiding more then three contiguous G bases
Selection of oligos with lower stability at the 5′ anti-sense terminus compared to the sense terminus. Duplexes with A-U or G-U base pairs at the 5′ end of the ant-sense strand and G-C base pairs at the 5′ end of the sense strand were preferred. Lower stability at the 5′ anti-sense terminus will favor the formation of an anti-sense RISC (RNA induced silencing complex)
Analyses were performed to ensure that the chosen sites do not target alternatively spliced exons and therefore would target all known variants of the genes studied.
   A standard synthetic RNAi reagent has the terminal two 3′ nucleotides as DNA (shown in with underlined lowercase letter), and the remainder being RNA for preferential uptake of the antisense strand into RISC (RNA induced silencing) complex.
   Using the criteria above, the following siRNA sequences were selected:

### Duplex name ACC NM_{—}001468_{—}15—GAGE#15

| Sense Sequence (5′-3′) (Position:249) | |
|---|---|
| GAACCAGCAACUCAACGUCAGGAtc | (SEQ ID NO: 1) |
| Antisense Sequence (5′-3′) (Position:273) | |
| GAUCCUGACGUUGAGUUGCUGGUUCCC | (SEQ ID NO: 2) |

Asymmetrical End Stability Difference: −0.41

### Duplex name ACC NM_{—}001468_{—}9—GAGE#9

Sense Sequence (5′-3′) (Position: 209)

| | |
|---|---|
| GUUCAGUGAUGAAGUGGAACCAGca | (SEQ ID NO: 3) |

AntiSense Sequence (5′-3′) (Position: 233)

| | |
|---|---|
| UGCUGGUUCCACUUCAUCACUGAACUG | (SEQ ID NO: 4) |

Asymmetrical End Stability Difference: −1.02
Duplex name: ACC NM_{—}133430_{—}2—XAGE1#2

| Sense Sequence (5′-3′) (Position: 186) | |
|---|---|
| GACAGAAGAAGAUCAGGAUACAGct | (SEQ ID NO: 5) |
| Antisense Sequence (5′-3′) (Position:210) | |
| AGCUGUAUCCUGAUCUUCUUCUGUCUG | (SEQ ID NO: 6) |

Asymmetrical End Stability Difference: −1.31

### Duplex name: ACC NM_{—}133430_{—}9—XAGE1#9

Sense Sequence (5′-3′) (Position: 395)

| | |
|---|---|
| AAGCUGAAACAACGCAAGCUGGUtt | (SEQ ID NO: 7) |

AntiSense Sequence (5′-3′) (Position: 419)

| | |
|---|---|
| AAACCAGCUUGCGUUGUUUCAGCUUGU | (SEQ ID NO: 8) |

Asymmetrical End Stability Difference: −0.03

### Duplex name: ACC NM_{—}005462_{—}19—MAGEC1

Sense Sequence (5′-3′) (Position: 2437)

| | |
|---|---|
| GGAGGACUCCCUCUCUCCUCUCCac | (SEQ ID NO: 9) |

Antisense Sequence (5′-3′) (Position: 2461)

| | |
|---|---|
| GUGGAGAGGAGAGAGGGAGUCCUCCCA | (SEQ ID NO: 10) |

Asymmetrical End Stability Difference: 1.18

### Duplex name: ACC NM_{—}005636 12—SSX4#12

| Sense Sequence (5′-3′) (Position: 586) | |
|---|---|
| CAAGGUCACCCUCCCACCUUUCAtg | (SEQ ID NO: 11) |
| Antisense Sequence (5′-3′) (Position: 610) | |
| CAUGAAAGGUGGGAGGGUGACCUUGAA | (SEQ ID NO: 12) |

Asymmetrical End Stability Difference: −0.01

### Duplex name: ACC NM_{—}005636_{—}19—SSX4#19

| Sense Sequence (5′-3′) (Position: 892 ) | |
|---|---|
| CUUGUGUAUCCAUGCACCUACCUca | (SEQ ID NO: 21) |
| Antisense Sequence (5′-3′) (Position: 916) | |
| UGAGGUAGGUGCAUGGAUACACAAGCC | (SEQ ID NO: 22) |

Asymmetrical End Stability Difference: −2.33

### Duplex name: ACC NM_{—}005362_{—}3 MAGEA

Sense Sequence (5′-3′) (Position: 1051)

| | |
|---|---|
| CCAGCUAUGUGAAAGUCCUGCACca | (SEQ ID NO: 23) |

Antisense Sequence (5′-3′) (Position: 1075)

| | |
|---|---|
| UGGUGCAGGACUUUCACAUAGCUGGUU | (SEQ ID NO: 24) |

Asymmetrical End Stability Difference: 0.94

### Duplex name: ACC NM_{—}016249_{—}3 MAGEC2#3

| Sense Sequence (5′-3′) (Position: 873 ) | |
|---|---|
| AGAUUACUUUCCUGUGAUACUCAag | (SEQ ID NO: 25) |
| Antisense Sequence (5′-3′) (Position:897) | |
| CUUGAGUAUCACAGGAAAGUAAUCUUU | (SEQ ID NO: 26) |

Asymmetrical End Stability Difference: −0.43

### Duplex name: ACC NM_{—}016249_{—}17 MAGEC2#17

| Sense Sequence (5′-3′) (Position:1545 ) | |
|---|---|
| CUCGAGGAACGUAGUGUUCUUUGca | (SEQ ID NO: 27) |
| Antisense Sequence (5′-3′) (Position:1569) | |
| UGCAAAGAACACUACGUUCCUCGAGCC | (SEQ ID NO 28:) |

Asymmetrical End Stability Difference: 1.51

### Duplex name: ACC NM_{—}001327_{—}7—NY-ESO-1 (CTAG1B)

| Sense sequence (5′-3′) (Position:451) | |
|---|---|
| GCUUCUGAAGGAGUUCACUGUGUcc | (SEQ ID NO: 13) |
| Antisense sequence (5′-3′) (Position:475) | |
| GGACACAGUGAACUCCUUCAGAAGCAC | (SEQ ID NO: 14) |

Asymmetrical End Stability Difference: 0

### Sequence of the negative control siRNAs used in this study (5′-3′):

Scrambled Sense:
(SEQ ID NO: 29)
CUU CCU CUC UUU CUC UCC CUU GUga
Scrambled Sense:
(SEQ ID NO: 30)
UCA CAA GGG AGA GAA AGA GAG GAA GGA
EGFP Sense:
(SEQ ID NO: 31)
ACCCUGAAGUUCAUCUGCACCACcg
EGFP Antisense:
(SEQ ID NO: 32)
CGGUGGUGCAGAUGAACUUCAGGGUCA

siRNA were purchased from IDT (Integrated DNA Technologies). The RNAs were resuspended in RNase-free Duplex Buffer (IDT) to 20 μM final concentration; vortexed thoroughly, microfuged and heated to 94° C. for 2 minutes, and allowed to cool to room temperature to ensure that the formation of duplexes. Once hydrated, duplexes were stored at −20° C. or −80° C. in aliquots. A scrambled universal negative control RNA duplex (DS Scrambled Neg) which is absent in human, mouse, and rat genomes, and siRNA specific to green fluorescent protein (GFP), and a positive control Dicer-Substrate RNA duplex (HPRT-S1 DS Positive Control) which targets a site in the HPRT (hypoxanthine guanine phosphoribosyltransferase 1) that is common between human, mouse, and rat and is prevalidated to give >90% knockdown of HPRT when transfected at 10 nM concentration were also purchased from IDT and used as negative and positive controls, respectively. The siRNA duplexes were used to transfect SK-MEL-37 and Du145 cells using Lipofectamine™ 2000 (Invitrogen) following the manufacturer's recommended protocols. Briefly, cells were seeded in 60 mm dishes in 4 ml of regular growth media without any antibiotics so the cells would be 50-60% confluent at the time of transfection. For transfection, 40 pmoles of siRNA were diluted in 500 μl Opti-MEM™ (Invitrogen). Eight μl of Lipofectamine™ 2000 were diluted in 500 μl Opti-MEM™ and incubated for 5 min at room temperature before mixing with the diluted siRNA. The siRNA-Lipofectamine™ 2000 mixture was incubated for 20 min at room temperature and then added to the cells. Twenty-four hours after incubation, the medium was replaced with growth medium (RPMI 10% fetal bovine serum). Cells were assayed 48-72 hours post-transfection.

### Cell Culture:

The cell lines SK-MEL-37, SK-MEL-119, SK-MEL-31, SK-MEL-124,SK-LC-5, PC3, Du145 and 22RV I were obtained from the cell culture bank of the New York Branch of the Ludwig Institute for Cancer Research. They were maintained in RPMI medium containing 10% fetal bovine serum (FBS) and non-essential amino acids.

### RNA Extraction, Reverse Transcription and RT-PCR:

Total RNA from the cell pellets was isolated using the RNeasy Mini Kit (Qiagen, Valencia, Calif.). RNA quantity was estimated by spectrophotometric analysis (Molecular Devices). A total of 0.5-1.0 μg of RNA was reverse transcribed into cDNA by using an Omniscript RT kit according to the manufacturer's protocol using oligo (dT)₁₈ primers. cDNAs were also prepared from a panel of 23 RNAs from normal tissues (Ambion, Austin, Tex.) and BD Biosciences (Palo Alto, Calif.). RT-PCR was undertaken with Jump-Start master mix (Sigma) plus 10 pmol of each of the following primers (predicted sizes of the PCR products in parenthesis):

GAGE F:
(SEQ ID NO: 15)
GACCAAGACGCTACGTAG (243 bp)
GAGE R:
(SEQ ID NO: 16)
CCATCAGGACCATCTTCA
XAGE1F:
(SEQ ID NO: 17)
TCCCAGGAGCCCAGTAATGGAGA (275 bp)
XAGE1R:
(SEQ ID NO: 18)
CAGCTTGTCTTCATTTAAACTTGTGGTTGC
XAGE1isoform1aF (plus XAGE1isoformR = 461 bp)
(SEQ ID NO: 33)
TTAAGGCACGAGGGAACCTCA C
XAGE1isoform1cF (plus XAGE1isoformR = 370 bp)
(SEQ ID NO: 34)
GGT ATC CGA GTC CCA GAA
XAGE1isoform1dF (plus XAGE1isoformR = 164 bp)
(SEQ ID NO: 35)
CCCAG GTGCTGGGAAGGGAAA
XAGE1isoformR
(SEQ ID NO: 36)
TGT GGT TGC TCT TCA CCT GC
MAGEA1F:
(SEQ ID NO: 27)
CGGCCGAAGGAACCTGACCCAG (421 bp)
MAGEA1R:
(SEQ ID NO: 38)
GCTGGAACCCTCACTGGGTTGCC
SSX4F:
(SEQ ID NO: 39)
AAATCGTCTATGTGTATATGAAGCT (278 and 414 bp)
SSX4R:
(SEQ ID NO: 40)
GGGTCGCTGATCTCTTCATAAAC
CTAG1BF:
(SEQ ID NO: 41)
CAGGGCTGAATGGATGCTGCAGA (332bp)
CTAG1BR:
(SEQ ID NO: 42)
GCGCCTCTGCCCTGAGGGAGG
MAGEC1F:
(SEQ ID NO: 43)
GACGAGGATCGTCTCAGGTCAGC (631 bp)
MAGEC1R:
(SEQ ID NO: 44)
ACATCCTCACCCTCAGGAGGG
MAGEC2F:
(SEQ ID NO: 45)
GGGAATCTGACGGATCGGA (355 bp)
MAGEC2:
(SEQ ID NO: 46)
GGAATGGAACGCCTGGAAC
ACTBF:
(SEQ ID NO: 19)
AAATCTGGCACCACACCTTC (644 bp)
ACTBR:
(SEQ ID NO: 20)
CACTGTGTTGCCGTACAGGT

The amplification involved three stages in which the annealing temperature was higher (60° C.) in the first ten cycles and reduced in two degrees in the following stage (ten cycles) and other two degrees in the last 15 cycles and involved an initial denaturation at 94° C. for 5min. Each cycle consisted of a denaturation step at 94° C. for 30 s, followed by 30 s at the annealing temperature and extension at 72° C. for 30 s followed by a final 7-min extension. Controls without DNA were carried out for each set of reaction. PCR products were loaded onto 2% agarose gels, stained with ethidium bromide and visualized by UV illumination.

### Quantitative Real-Time Reverse Transcription-PCR:

cDNA samples were run in duplicate for the genes of interest and for the reference gene within the same experiment using the Applied Biosystem apparatus 7500 Fast Real-Time PCR system and Taqman platform (Applied Biosystems, Foster City, Calif.). TFRC was amplified as an internal reference gene. The PCR primers and probes for all tested genes (MAGEA3, GAGE, SSX4, NY-ESO-1, MAGEC1, MAGEC2, XAGE1) and internal control gene (TFRC) were purchased from Applied Biosystems. Primers used for PCR amplification were chosen to encompass intron between exon sequences to avoid amplification of genomic DNA (Applied Biosystems,). XAGE1 primers for real-time PCR were selected to amplify all three XAGE1 isoforms (NM_{—}001097591, NM_{—}001097592 and NM_{—}001097593). Likewise, GAGE primers were selected to amplify GAGE1, 2, 7, 7B, 8, 6, 5 and 4. The gene-specific probes were labeled with the reporter dye 6-FAM at the 5′-end. The TFRC probe was labeled with a reporter dye (VIC) to the 5′-end of the probe and all probes had minor groove binder/nonfluorescent quencher at the 3′-end of the probe (Applied Biosystems). The PCR conditions were 95° C. for 10 minutes followed by 40 cycles at 95° C. for 15 seconds and 60° C. for 1 minute. Duplicate C_{T}S were averaged for each sample. Relative quantification of gene expression (relative amount of target RNA) was determined using the equation 2^{-ΔΔCT}.

Migration and Invasion ssays:

Cell migration and invasion were assessed in 12-well Boyden Chambers (BD Biosciences, San Diego, Calif.) according to the protocol of the manufacturer. Invasion assays were carried out in chamber equipped with an 8 μm polycarbonate membrane coated with Matrigel. Briefly, cells were serum-starved for 2 hr, and 500 μl containing 25,000 cells in medium supplemented with 1% FBS were loaded into the upper chamber. The lower chamber contained medium supplemented with 10% FBS as chemoattractant for SK-MEL-37 and with medium supplemented with 10% FBS and 100 ng/ml hEGF for Du145. Cells were incubated at 37° C. overnight, fixed in 10% formalin for 20 min and stained with 0.2% crystal violet (Fisher Scientific, Pittsburgh, Pa.). Non-invading cells on the top of the membrane were wiped off using cotton swabs, and invading cells affixed to the underside of the membranes on each insert were counted at 100 x magnification in 10 random areas. The migration assay was done in a similar fashion except the 8.0-μm pore size membrane inserts were not coated with Matrigel. Results were expressed as mean±SE.

### Cell Viability Assay (Colony Formation Assay):

At 48 h after transfection with each siRNA, cells were trypsinized, counted and 1,000 cells were seeded in duplicate in 6-well plates and allowed to form colonies for 2 weeks. The colonies were fixed with 10% formallin and stained with 0.1% crystal violet (Fisher Scientific, Pittsburgh, Pa.). The number of colonies with 30 cells or larger than 1 mm in diameter in each well was counted.

### Anchorage-Independent Growth in Soft Agar

A total of 5×10³ cells transfected with CT-specific or non-targeting siRNAs were plated in 0.35% agar in lx DMEM, over a layer of 0.5% agar/lx RPMI 10% FBS, on 6-well plates. The immobilized cells were grown for 14-21 days in the presence of RPMI supplemented with 10% FCS in a humidified chamber at 37° C. with 5% CO₂. Plates were stained with 0.005% crystal violet and the number of the colonies were registered.

### Western Blotting Analyses

Cells were harvested and washed with cold phosphate-buffered saline solution, and total proteins were extracted in the extraction buffer (50 mM Tris-Cl pH 7.4, 0.15 M NaCl, 2 mM EDTA 1% NP40), containing protease inhibitors (Protease Inhibitors Cocktail, Roche, Indianapolis, Ind.). Equal amounts of protein (20 μg per lane) were mixed with an equal volume of 2× loading buffer (125 mM Tris-HCl pH 6.8, 4% SDS, 10% glycerol, 0.006% bromophenol blue, 2%(-mercaptoethanol), incubated at 95° C. for 3 mM, and loaded in 10% SDS Bis-Tris gels (Invitrogen, Carlsbad, Calif.). After electrophoresis, proteins were transferred to nitrocellulose membranes. The membranes were blocked by incubation in PBST (PBS 0.1% Tween 20) 3% bovine serum albumin (BSA) for 1 h, then incubated with the primary antibody overnight at 4° C. in PBST 1% BSA. After washing four times in PBST, the membranes were incubated either with peroxidase-conjugated anti-rabbit or anti-mouse IgG (Jackson Immunoresearch, Bar Harbor, ME) for 1 h at room temperature. Antibody binding was detected using the system Western Lightening Chemiluminescence Reagent Plus (Perkin Elmer, Emeryville, Calif.). The antibodies used were: a monoclonal anti-GAGE (611746, BD Transduction Laboratories, San Diego, Calif.) and a rabbit polyclonal anti-actin (20-33, Sigma-Aldrich, St. Louis, Mo.).

### Statistical Analyses:

Student's t-test was used to compare the differences between groups. A p-value <0.05 was considered statistically significant.

### Example 2

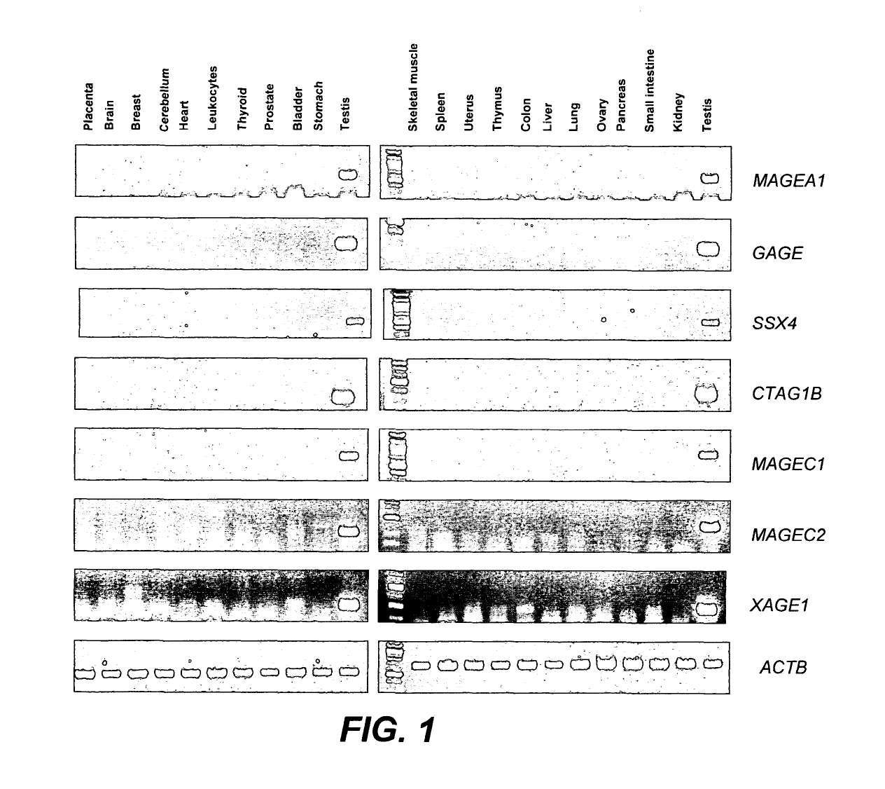

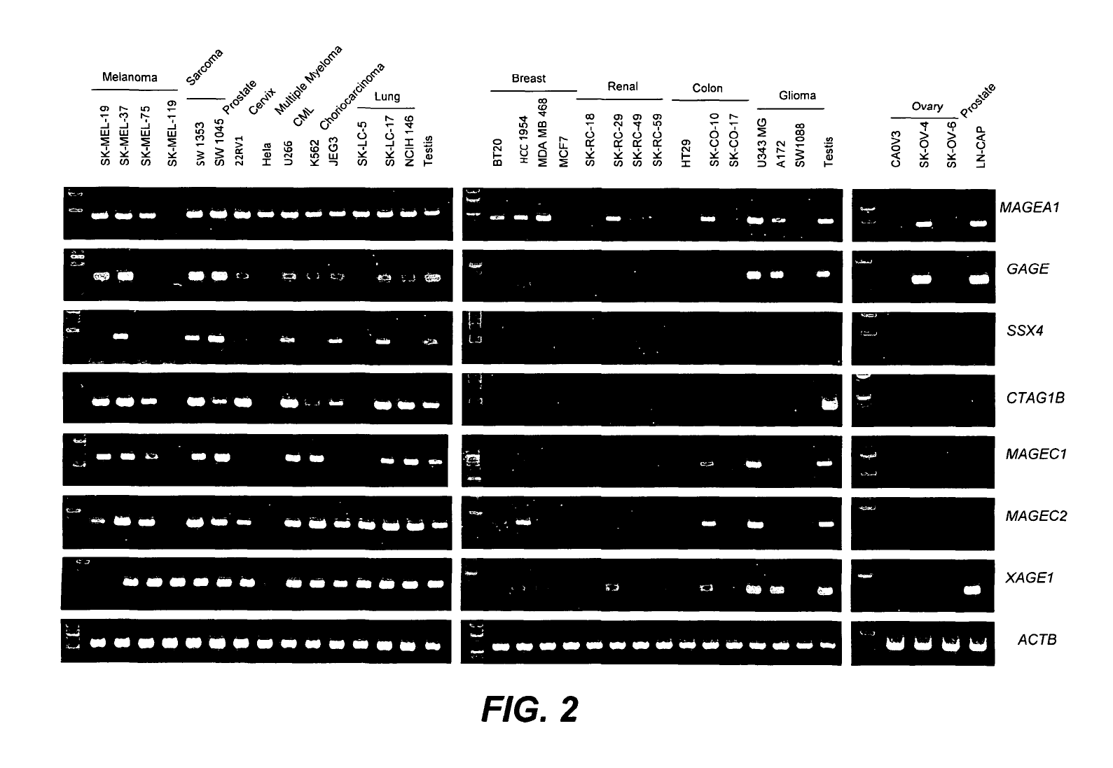

To first assess the potential utility of MAGEA1, GAGE, SSX, CTAG1B, MAGEC1, MAGEC2 and XAGE1 as therapeutic targets, we examined their expression in a variety of normal tissues by RT-PCR (FIG. 1). The apparent absence of expression MAGEA1, GAGE, SSX4, CTAG1B, MAGEC1, MAGEC2 in normal tissue except testis and the very restricted expression of XAGE1 is consistent with their classification as a Cancer testis (CT) antigen and encouraging in terms of its utility as a target. We also have determined the expression of these genes by RT-PCR in a set of 32 cancer cell lines derived from tumors of different origins (FIG. 2). We found that most of them were expressed in melanoma cell lines and therefore we decided to investigate whether MAGEA1, GAGE, SSX, CTAG1B, MAGEC1, MAGEC2 and XAGE1 might be directly related to the malignant properties of cancer cell lines derived from melanoma.

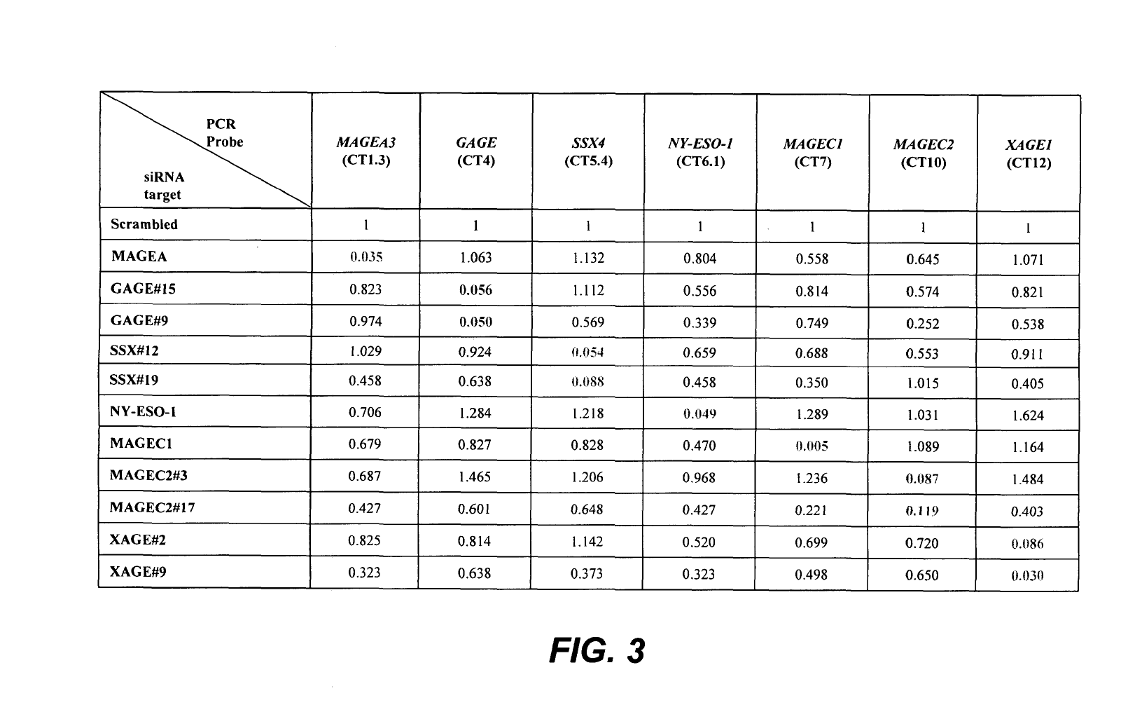

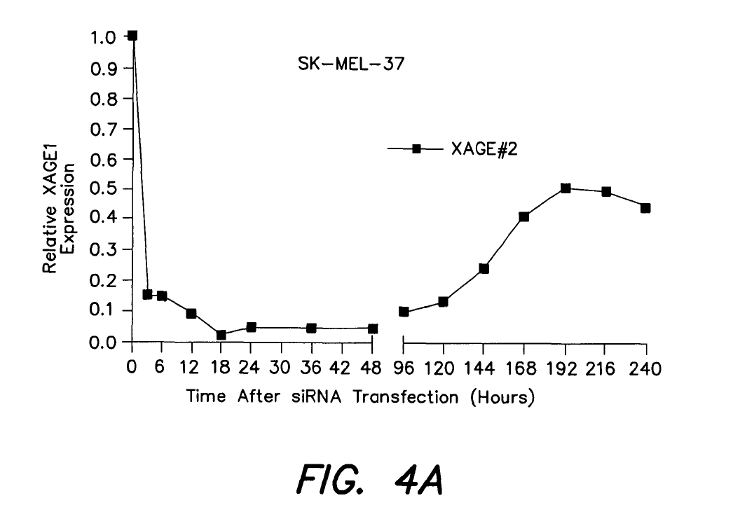

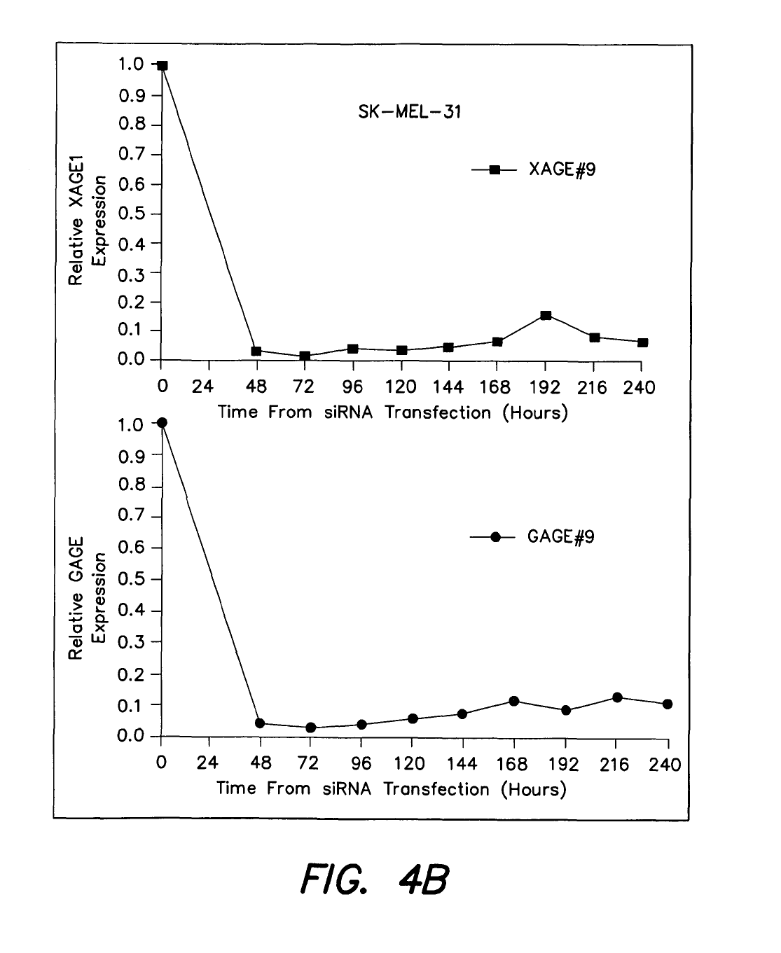

To this end, we used small interference RNAs (siRNAs) to reduce MAGEA1, GAGE, SSX, CTAG1B, MAGEC1, MAGEC2 and XAGE1 mRNA levels in malignant cell lines. We designed and tested siRNAs specific to these genes. We also used a scrambled siRNA (IDT, Coralville, Iowa) as negative controls. These siRNA duplexes targeting the coding regions of the different CT-X were individually introduced into the SK-MEL-37 melanoma cell line and the effect on mRNA level was examined by real-time quantitative RT-PCR analysis 48 hours post transfection. All siRNA duplexes examined produced a 91-99% reduction in CT-X mRNA compared with the control sample transfected with scrambled siRNA as negative control (FIG. 3). In addition, we analyzed the effects of each siRNA duplex on the mRNA level of other CT-X, and little to no effect was observed compared with the scrambled control siRNA, suggesting that the effects of the 27mer siRNAs on these genes were sequence-specific. For XAGE and GAGE duplexes, we also examined the kinetics of gene silencing and analyzed the levels of mRNA at 3, 6, 12, 18, 24, 36 and 48 hours after transfection (FIG. 4). Around 75-80% mRNA reduction could be observed as early as three hours after transfection and around 2 fold knock down was still detectable 10 days after transfection in SK-MEL-37 (FIG. 4). The same experiment in a melanoma cell line that presents a lower growth rate (SK-MEL-31), revealed that more than 10-fold knock down was still present 10 days after transfection (FIG. 4).

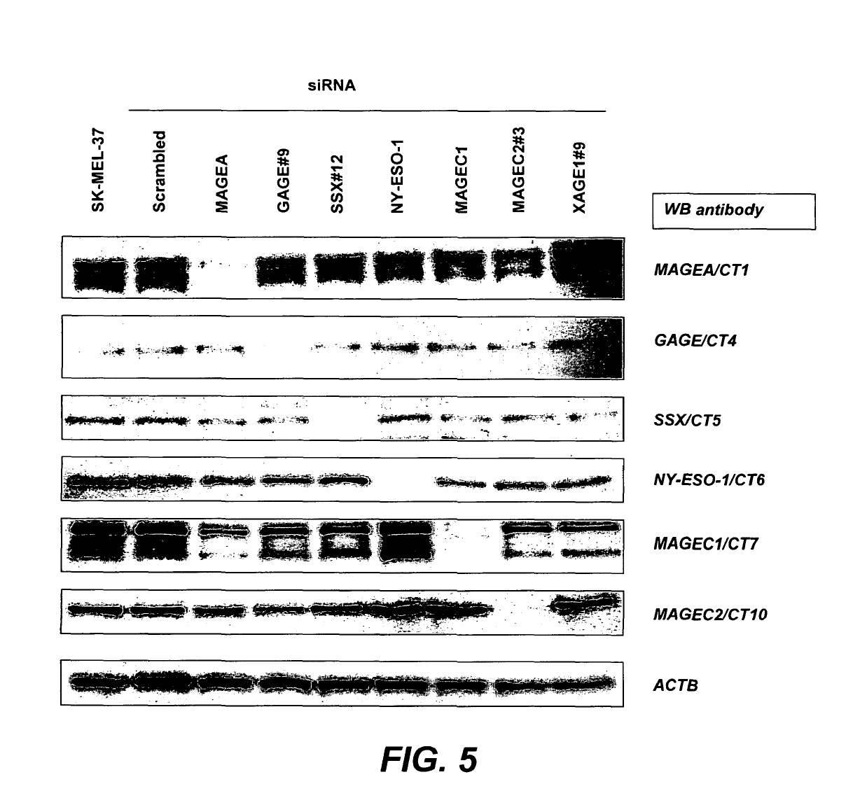

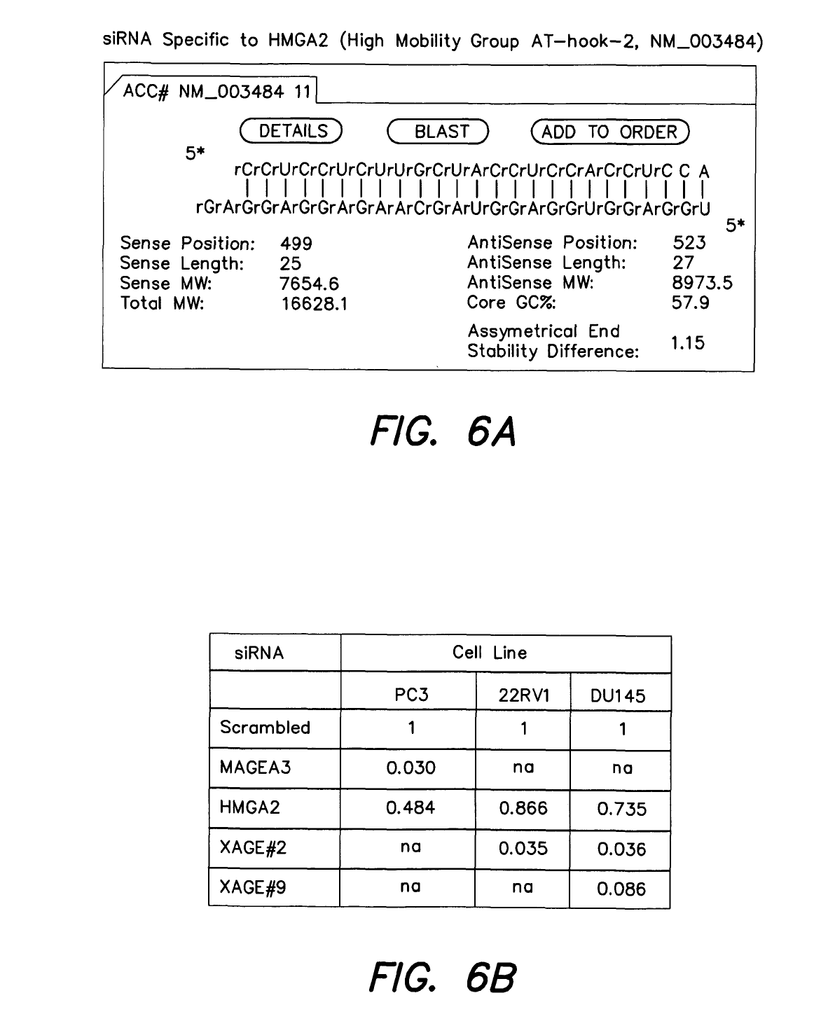

However, a siRNA specific to HMGA2, designed with the same online tools available at idtdna.com/Scitools/Applications/RNAVRNALaspx, but without taking into consideration any optimization criteria, failed to produce gene knock down in three different cancer cell lines (PC3, 22RV1, DU145) while in the same experiment, siRNAs specific to CT-X independently transfected produced very efficient knock down, showing that the algorithm available at this site not always produce efficient reagents (FIG. 5).

Western blot analysis was used to examine the effect of the specific siRNAs on CT-X expression at the protein level, in the cases where antibodies are available (MAGEA, GAGE, SSX, NY-ESO-1, MAGEC1 and CT10). We analyzed the effects siRNAs 72 h after transfection and we were able to show that in all cases, reduction in protein levels to almost complete depletion was present at this time point (FIG. 5).

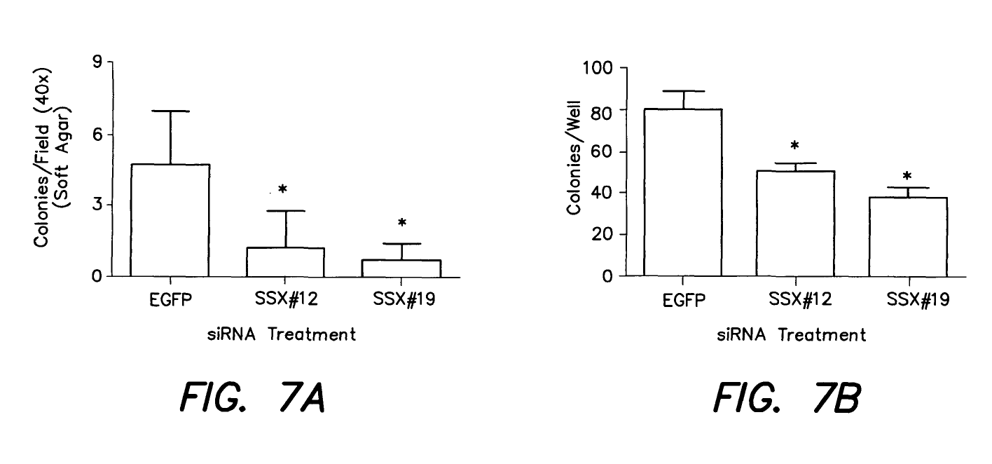

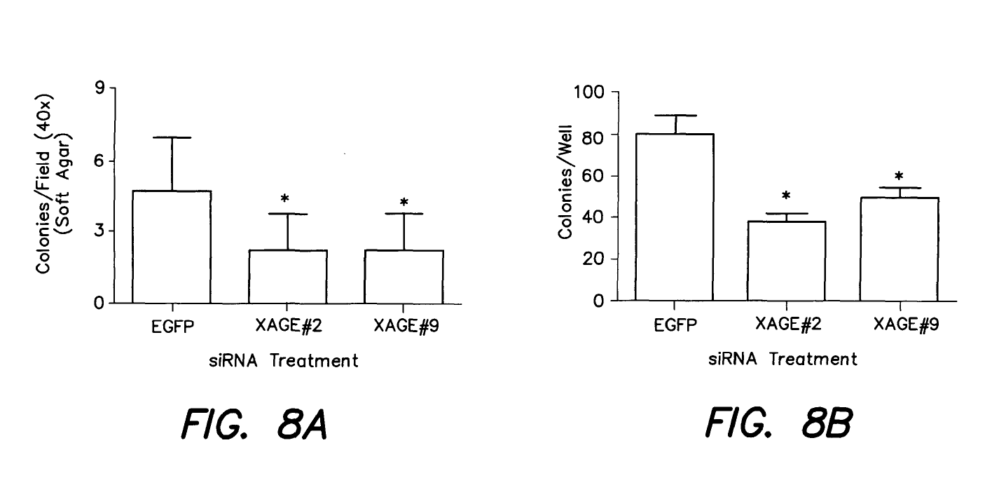

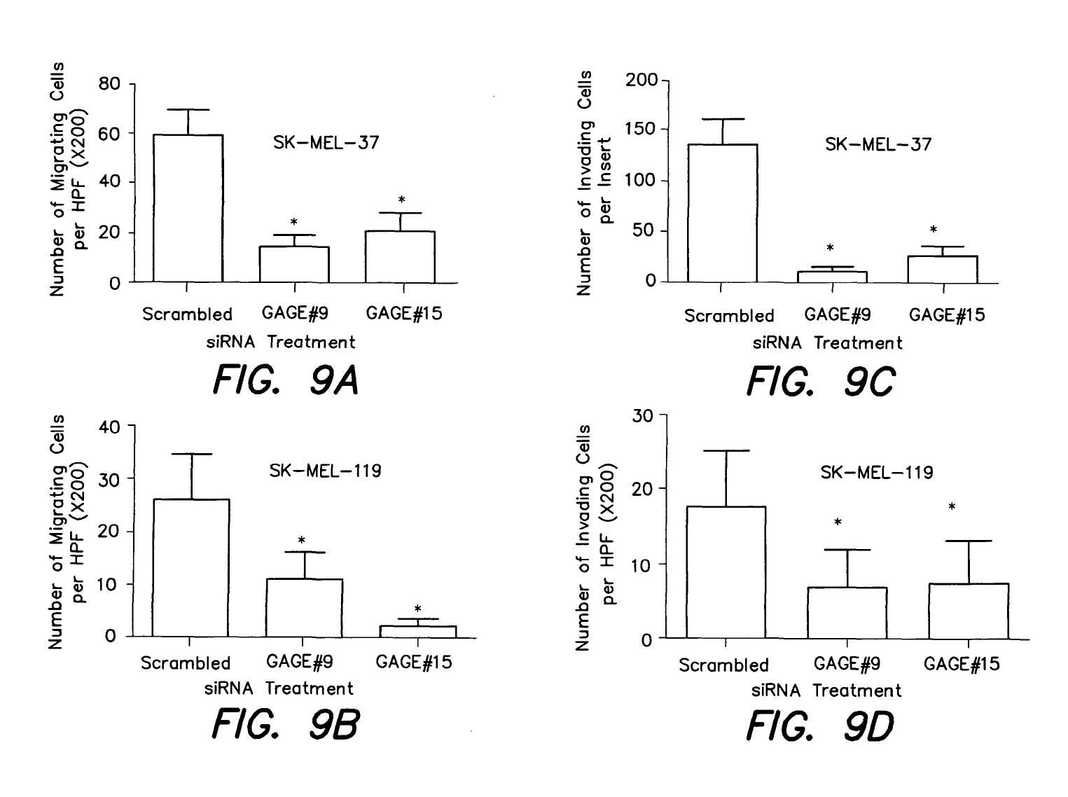

To investigate the biological results of depletion of CT-X by RNAi, we examined growth and migratory phenotypes of the melanoma cell line SK-MEL-37, which expresses high levels of the seven CT antigens studied. First, we analyzed the ability of the siRNA-treated cells to form colonies between 10 and 14 days after transfection. The clonogenic assay, has traditionally been considered to be the optimal method for determining survival after cytotoxic treatment, such as radiation. This assay relies on the ability of cells to form viable colonies derived from a single cell. In this colony formation assay, only 5-10% of control cells gave rise to colonies (plating efficiency). We also tested the ability of the transfected cells to form colonies in soft agar. Depletion of SSX4 and XAGE1 significantly reduced the colony-forming ability of SK-MEL-37 cells to 50% or less of control levels (FIGS. 7 and 8, respectively).

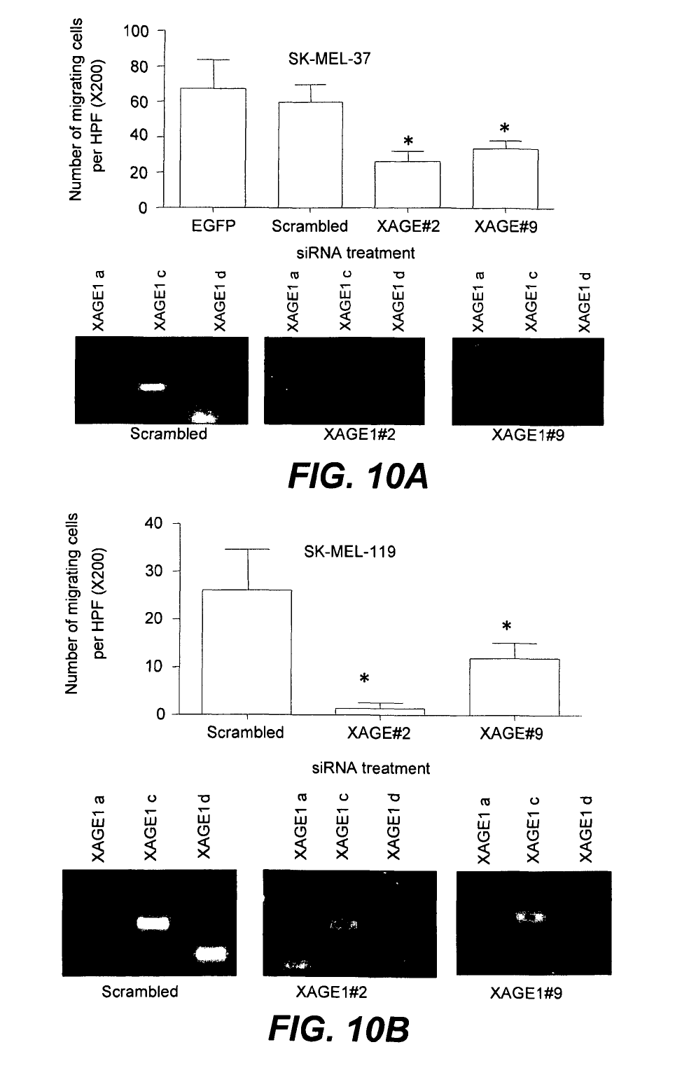

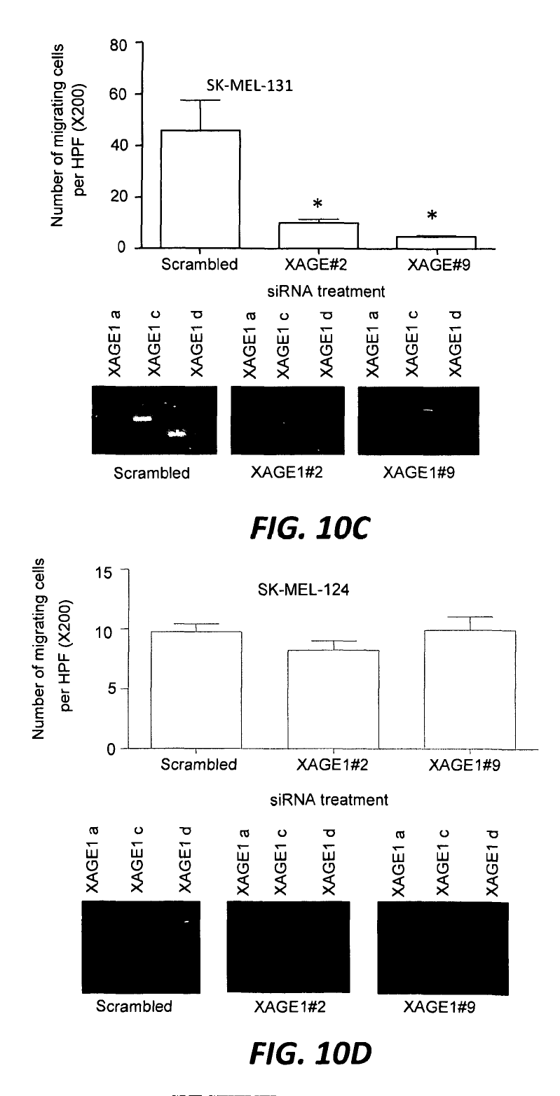

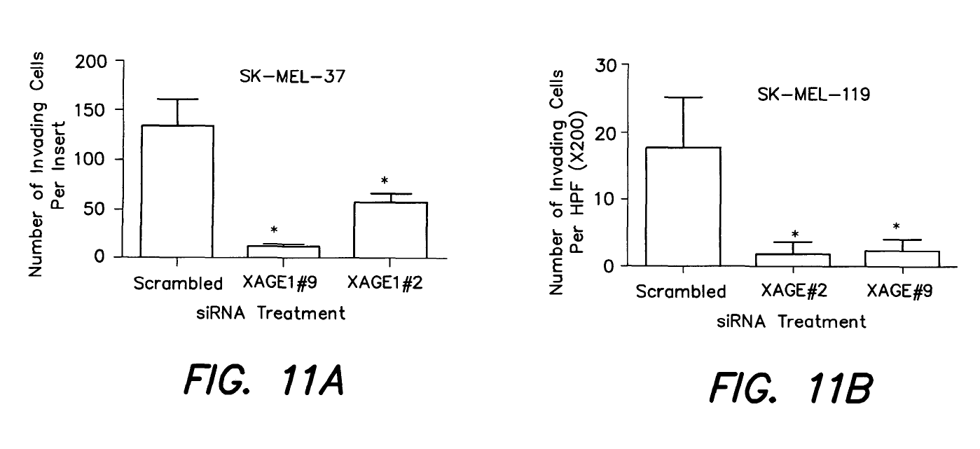

To determine the possible role of CT-X in the migration and invasion properties of melanoma cells we used a transwell migration and invasion assays. siRNAs specific to GAGE (FIG. 9) and XAGE1 (FIG. 10) significantly inhibited migration and invasion of melanoma cells. For XAGE1, we also tested additional cell lines that express high levels of this gene (SK-MEL-119 and SK-MEL-131) and the same effect was observed, but in a melanoma cell line that do not express XAGE 1, the siRNAs specific to this gene had no effect on cell migration (FIGS. 10 and 11).

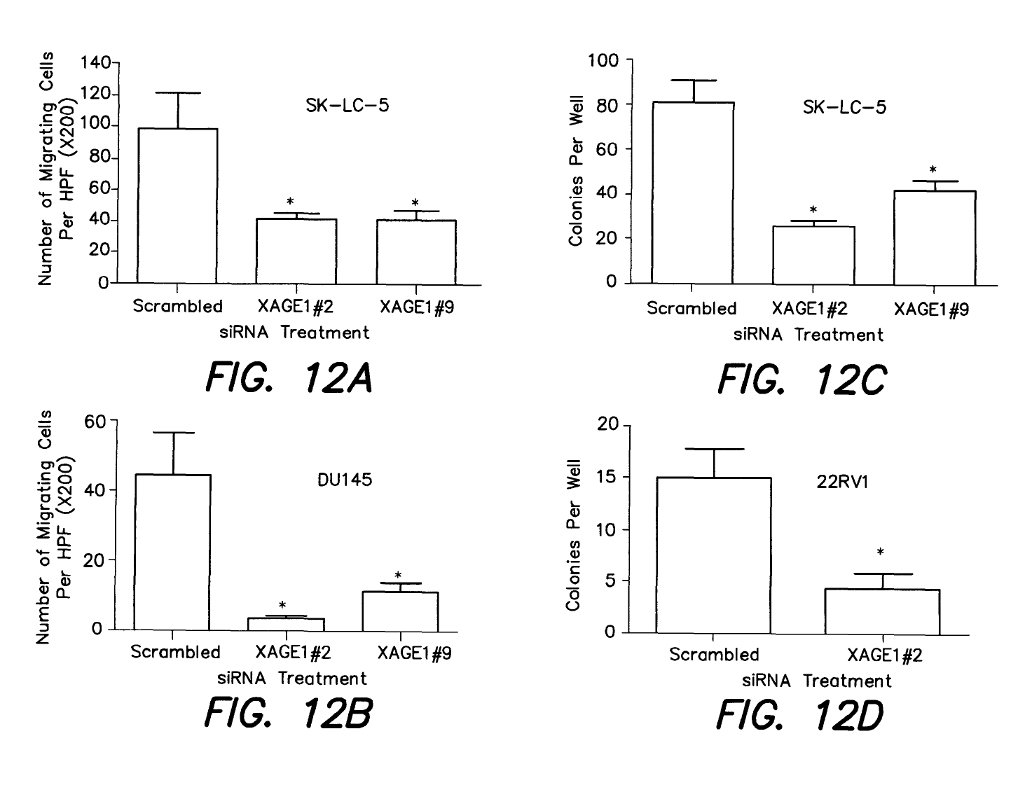

FIG. 12 shows that the effect of XAGE1 knockdown on colony formation and cell migration can also be observed in prostate (22RV1 and DU145) and lung cancer (SK-LC-05) cell lines.

Overall, these results suggest that this level of inhibition on SSX, XAGE1 and GAGE expression in cancer cell lines is sufficient to interfere with tumor cell migration and reduce cell viability. We demonstrate that the observed RNAi-induced phenotype is probably a result of the suppression of CT-antigen expression and is an off-target effect, which arise from unintended interactions, whether dependent on nucleotide sequence or not, between the silencing molecules and various cellular components. The finding that multiple siRNAs that target different regions of the same gene, used in this study for XAGE1, GAGE and SSX, have the same phenotypic effect, offer the most convincing controls that these effects are indeed dependent on their depletion.

### Example 3

To analyze the expression of XAGE1 and GAGE in tumors we undertook a meta-analysis of microarray data deposited in the Oncomine website (oncomine.org). We found XAGE1 to be overexpressed in different tumor types, as compared with the respective normal tissues, among them, tumors of the prostate, melanoma, breast and pancreas. We found GAGE to be overexpressed in melanoma, and tumors of the prostate and lung.

From the analysis of the microarray data, among the tumor types in which these two genes were found to be overexpressed, we elected to investigate whether XAGE1 and GAGE might be directly related to the malignant properties of cell lines derived from prostate cancer and melanoma.

We used small interfering RNAs (siRNAs) to reduce XAGE1 and GAGE mRNA levels in malignant cell lines. We designed and tested siRNAs specific to XAGE1 and GAGE. We also used a scrambled siRNA (IDT, Coralville, Iowa) and siRNAs specific to other CT antigens (NY-ESO-1, SSX and MAGEC1) as negative controls. The levels of all genes tested SK-MEL-37 were reduced at least 95% 48 h after transfection, as compared with the levels in the cells transfected with the scrambled siRNA.

We tested if this procedure had effects on cell migration, invasion and in cell viability, as assessed by a colony formation assay. We determined that the treatment of SK-MEL-37 cells with siRNAs specific to XAGE1, SSX and MAGEC1 reduced the levels of the respective mRNAs and that knock down of XAGE1 had a profound effect on cell migration in a trans-well assay, but not the ones specific to SSX and MAGEC1 or the scrambled siRNA.

We determined the effect of treatment of SK-MEL-37 melanoma cells with siRNAs specific to GAGE were effective in decreasing GAGE mRNA levels and also cell migration. XAGE1 was used as a positive control and scrambled siRNA as a negative control in this experiment.

We determined that the effect of XAGE1 knockdown on cell migration can also be observed in prostate and breast cancer cell lines. We treated the DU145 prostate cancer cell line with XAGE1 specific siRNA and also with scrambled siRNA. We observed that transwell migration and also invasion through a Matrigel layer were significantly decreased by XAGE1 siRNA. We also determined the effects of siRNA specific to XAGE1 in knocking down XAGE1 levels and in decreasing MDA-MB-231 breast cancer cell migration in the transwell migration assay.

We determined the effect of XAGE1 and GAGE knockdown on cell viability. We found that treatment of both a prostate cancer cell line (22RV-1) and a melanoma cell line (SK-MEL-37) with XAGE1 specific siRNA resulted in a reduction in cell viability. We determined that GAGE knockdown also decreased cell viability in SK-MEL-37 cell line. In both experiments, knockdown also decreased cell viability in SK-MEL-37 cell line. In both experiments, siRNA specific to another CT antigen (NY-ESO-1) and scrambled siRNA were used as negative controls.

Overall, these results indicate that this level of inhibition on XAGE1 and GAGE expression in prostate cancer and melanoma cell lines is sufficient to interfere with tumor cell migration and reduce cell viability.

### Example 4

In vivo experiments demonstrate the role of XAGE1 and GAGE in tumor growth and metastasis, and involve delivery of multivalent siRNAs, which are developed based on the active 27-mers specific to GAGE and XAGE1 disclosed herein, by means of antibodies, aptamers, or other suitable molecules to treat cancer.

### Example 5

Multivalent siRNAs, which are developed based on the active 27-mers specific to GAGE and XAGE1 disclosed herein, are conjugated to PSMA aptamers or PSMA antibodies for use in animal models of prostate cancer.

### Example 6

Assessment of the effects of XAGE1 and GAGE knockdown in models of tumor growth and metastasis (for melanoma, prostate and breast cancer).

Plasmid- and viral vector-based constitutive expression of shRNAs often results in stable and efficient suppression of target genes. However, the inability to adjust levels of suppression has limited the analysis of genes essential for cell survival, cell cycle regulation, and cell development. Besides, suppression of a gene for longer periods may result in nonphysiological responses. This problem can be circumvented by generating inducible regulation of RNAi in mammalian cells. For these reasons, a plasmid vector-mediated tetracycline-inducible short-hairpin RNA (shRNA) expression system is used to evaluate the role of XAGE1 and GAGE using previously established mouse models for tumor growth and metastasis. In this system, RNAi expression follows a stringent dose- and time-dependent kinetics of induction with undetectable background expression in the absence of the inducer. After analyzing several different tetracycline-inducible systems for shRNA expression, Clontech's Tet-On Advanced Inducible Gene Expression System (Urlinger et al., Proc Natl Acad Sci U S A. 2000 Jul. 5; 97(14):7963-8) is used. This system consists of 2 components that have been optimized for use in mammalian cells: a regulator vector, pTet-On-Advanced that expresses the tetracycline-controlled transactivator and a response vector, containing an improved tetracycline response element (TRE) within the promoter that controls expression of the shRNA. In this system, a stable cell line expressing the Tet-On Advanced transactivator is generated. The inducer doxycycline (Dox, a tetracycline derivative) controls the system in a dose-dependent manner, allowing a precise modulation of the expression levels. The response vector is a retroviral micro-RNA-based plasmid that produces potent, stable and regulatable gene knock down in cultured cells and animals (pTMP) (Dickins et al., Nat Genet. 2005 November; 37(11):1289-95).

Inducible Expression of shRNA:

Stable pTet-On-Advanced cell lines (clones) are generated and tested. For example, the ability of pTet-On-Advanced clones to induce the expression of reporter plasmid containing TREs is tested. Generation of the pTMP constructs with the chosen siRNAS (21 or 22mers) selected from within the active 27-mer duplexes used in the transient transfection experiments. At least two 22-mers are tested for their ability of knocking down gene expression.

Stable pTet-On-Advanced clones are generated for melanoma cell lines (SK-MEL-37, and LM-MEL-34) and a prostate cell line (DU145). pTMP shRNA constructs are developed for XAGE1, GAGE, MAGEA, CT7 and NY-ESO-1. Transfer of pTMP-shRNA constructs and empty pTMP into pTet-On-Advanced clones is accomplished by retroviral delivery to create double-stable cell lines. Double stable cell lines are developed for XAGE1, GAGE, MAGEA, CT7 and NY-ESO-1. Induction of shRNA expression for each gene and associate biological effects (proliferation rates, migration and invasion capabilities) are tested in vitro.

### Example 7

The double-stable cell lines generated according to the procedure set forth in Example 7 are used in experiments that permit dose- and time-dependent suppression of XAGE1 and GAGE gene expression (and empty vector as negative control) to evaluate tumor growth and metastasis. Tumor growth is evaluated by subcutaneous (s.c.) injections of tumor cells (melanoma, prostate and breast cancer) in the flanks of nude mice followed by serial measurements of tumor volumes.

The ability to metastasize is evaluated by different assays depending on the tumor type analyzed and include, for example, injection of tumors into footpads of nude mice to evaluate the ability to metastasize from footpad to lymph nodes, assessment of development of spontaneous lung metastasis after subcutaneous injections of tumor cells in nude mice, and injection of tumor cells through the tail vein and evaluation of lung, liver and kidney metastases.

### REFERENCES

1. Kim D H, Rossi J J: Strategies for silencing human disease using RNA interference. Nat Rev Genet 2007, 8(3):173-184.
2. Amarzguioui M, Lundberg P, Cantin E, Hagstrom J, Behlke M A, Rossi J J: Rational design and in vitro and in vivo delivery of Dicer substrate siRNA. Nat Protoc 2006, 1(2):508-517.
3. Kurreck J: siRNA Efficiency: Structure or Sequence-That Is the Question. J Biomed Biotechnol 2006, 2006(4):83757.
4. Patzel V: In silico selection of active siRNA. Drug Discov Today 2007, 12(3-4):139-148.

### Example 8

We designed a second siRNA (XAGE1#9) to exclude certain off-target effects of the first XAGE1-specific siRNA (XAGE1#2). XAGE1#2 has sense and antisense start positions of 186 and 210, respectively in NM_{—}133430. XAGE1#9 has sense and antisense start positions of 395 and 419, respectively in NM_{—}133430. We determined that silencing of

XAGE1 using XAGE1#2 and XAGE1#9 27-mer siRNAs equally reduces viability and transwell migration of the SK-MEL-37 melanoma cell line and equally reduces viability and transwell migration of the SK-LC-5 NSCLC cancer cell line. We also determined that silencing of XAGE1 using XAGE1#2 and XAGE1#9 equally reduces transwell migration of Du145 prostate cancer cell-line. In addition, we determined that treatment of SK-MEL-124, a XAGE1 negative melanoma cell line, with XAGE1#2 and XAGE1#9 siRNAs does not affect transwell migration

### Example 9

We designed a second siRNA (GAGE1#9) to exclude certain off-target effects of the first GAGE1-specific siRNA (GAGE1#15). GAGE1#15 has sense and antisense start positions in NM_{—}001468 of 249 and 273, respectively. GAGE1#9 has sense and antisense start positions in NM_{—}001468.of 209 and 233, respectively We determined that treatment of SK-MEL-37 cells with either GAGE1#15 or GAGE1#9 significantly reduces GAGE protein levels and equally reduces transwell migration of SK-MEL-37 melanoma cell-line.

### Example 10

We designed a second siRNA (SSX4#12) to exclude certain off-target effects of the SSX4-specific siRNA (SSX4#12). SSX4#12 has sense and antisense start positions in NM_{—}005636 of 586 and 610, respectively. SSX4#19 has sense and antisense start positions in NM_{—}005636 of 892 and 916, respectively We determined that treatment of SK-MEL-37 cells with either SSX4#12 or SSX4#19 significantly inhibits colony formation in soft agar and clonogenic survival of the SK-MEL-37 cell line.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only. All references described herein are incorporated by reference for the purposes described herein.

Moreover, this invention is not limited in its application to the details of construction and the arrangement of components set forth in the disclosed description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

All references disclosed herein are incorporated by reference in their entirety, and particularly for the purposed cited herein.

What is claimed is:

1. An isolated small interfering nucleic acid comprising a nucleic acid consisting of the sequence set forth in SEQ ID NO. 2; or the sequence set forth in SEQ ID NO. 4; or the sequence set forth in SEQ ID NO. 6; or the sequence set forth in SEQ ID NO. 8; or the sequence set forth in SEQ ID NO. 10; or the sequence set forth in SEQ ID NO. 12; or the sequence set forth in SEQ ID NO. 14; or the sequence set forth in SEQ ID NO. 22; or the sequence set forth in SEQ ID NO. 24; or the sequence set forth in SEQ ID NO. 26; or the sequence set forth in SEQ ID NO. 28.

2-11. (canceled)

12. The isolated small interfering nucleic acid of claim 1, having a sense strand consisting of the sequence set forth in SEQ ID NO. 1 and an antisense strand consisting of the sequence set forth in SEQ ID NO. 2; or a sense strand consisting of the sequence set forth in SEQ ID NO. 3 and an antisense strand consisting of the sequence set forth in SEQ ID NO. 4; or a sense strand consisting of the sequence set forth in SEQ ID NO. 5 and an antisense strand consisting of the sequence set forth in SEQ ID NO. 6; or a sense strand consisting of the sequence set forth in SEQ ID NO. 7 and an antisense strand consisting of the sequence set forth in SEQ ID NO. 8; a sense strand consisting of the sequence set forth in SEQ ID NO. 9 and an antisense strand consisting of the sequence set forth in SEQ ID NO. 10; or a sense strand consisting of the sequence set forth in SEQ ID NO. 11 and an antisense strand consisting of the sequence set forth in SEQ ID NO. 12; or a sense strand consisting of the sequence set forth in SEQ ID NO. 13 and an antisense strand consisting of the sequence set forth in SEQ ID NO. 14; or a sense strand consisting of the sequence set forth in SEQ ID NO. 21 and an antisense strand consisting of the sequence set forth in SEQ ID NO. 22; or a sense strand consisting of the sequence set forth in SEQ ID NO. 23 and an antisense strand consisting of the sequence set forth in SEQ ID NO. 24; or a sense strand consisting of the sequence set forth in SEQ ID NO. 25 and an antisense strand consisting of the sequence set forth in SEQ ID NO. 26; or a sense strand consisting of the sequence set forth in SEQ ID NO. 27 and an antisense strand consisting of the sequence set forth in SEQ ID NO. 28.

13-22. (canceled)

23. The isolated small interfering nucleic acid of claim 1, wherein the isolated small interfering nucleic acid is a 27-mer siRNA.

24. The isolated small interfering nucleic acid of claim 1, wherein the isolated small interfering nucleic acid is a short-hairpin RNA.

25. A composition comprising the isolated small interfering nucleic acid of claim 1, optionally further comprising a transfection reagent.

26. (canceled)

27. A method for inhibiting expression of a cancer testis antigen in a cell, comprising: contacting the cell with the composition of claim 25.

28. The method of claim 27, wherein the contacting results in uptake of the isolated small interfering nucleic acid in the cell.

29. A pharmaceutical formulation comprising: (i) an isolated small interfering nucleic acid of claim 1 and (ii) a pharmaceutically acceptable carrier.

30. A pharmaceutical kit comprising (i) a container housing the pharmaceutical formulation of claim 29 and (ii) instructions for administering the pharmaceutical formulation to a individual.

31. A kit comprising (i) a container housing the composition of claim 25, (ii) instructions for transfecting a cell with the small interfering nucleic acid, and optionally (iii) a container housing a transfection reagent.

32. A method for inhibiting viability, invasion, colony formation, and/or migration of a cancer cell comprising contacting the cancer cell with an effective amount of a molecule capable of inhibiting expression of XAGE, a molecule capable of inhibiting expression of GAGE, and/or a molecule capable of inhibiting expression of SSX.

33. The method of claim 32, wherein the molecule capable of inhibiting expression of XAGE is or encodes a small interfering nucleic acid capable of inhibiting expression of XAGE, the molecule capable of inhibiting expression of GAGE is or encodes a small interfering nucleic acid capable of inhibiting expression of GAGE, and/or the molecule capable of inhibiting expression of SSX is or encodes a small interfering nucleic acid capable of inhibiting expression of SSX.

34. The method of claim 33, wherein the small interfering nucleic acid capable of inhibiting expression of GAGE comprises a nucleic acid sequence consisting of SEQ ID NO. 2 or SEQ ID NO. 4; and/or wherein the small interfering nucleic acid capable of inhibiting expression of XAGE comprises a nucleic acid sequence consisting of SEQ ID NO. 6 or SEQ ID NO. 8; and/or wherein the small interfering nucleic acid capable of inhibiting expression of SSX comprises a nucleic acid sequence consisting of SEQ ID NO. 12 or SEQ ID NO. 22; and/or wherein the small interfering nucleic acid capable of inhibiting expression of GAGE is a duplex having a sense strand consisting of SEQ ID NO. 1 and an antisense strand consisting of SEQ ID NO. 2; and/or wherein the small interfering nucleic acid capable of inhibiting expression of GAGE is a duplex having a sense strand consisting of SEQ ID NO. 3 and an antisense strand consisting of SEQ ID NO. 4; and/or wherein the small interfering nucleic acid capable of inhibiting expression of XAGE is a duplex having a sense strand consisting of SEQ ID NO. 5 and an antisense strand consisting of SEQ ID NO. 6; and/or wherein the small interfering nucleic acid capable of inhibiting expression of XAGE is a duplex having a sense strand consisting of SEQ ID NO. 7 and an antisense strand consisting of SEQ ID NO. 8; and/or wherein the small interfering nucleic acid capable of inhibiting expression of SSX is a duplex having a sense strand consisting of SEQ ID NO. 11 and an antisense strand consisting of SEQ ID NO. 12; and/or wherein the small interfering nucleic acid capable of inhibiting expression of SSX is a duplex having a sense strand consisting of SEQ ID NO. 21 and an antisense strand consisting of SEQ ID NO. 22.

35-42. (canceled)

43. The method of claim 33, wherein the small interfering nucleic acid capable of inhibiting expression of GAGE, XAGE or SSX is a 27-mer siRNA or a small hairpin RNA.

44-45. (canceled)

46. The method of claim 32, wherein the cancer cell is in vitro, or wherein the cancer cell is in a subject in need of a treatment effective to inhibit viability, invasion, colony formation and/or migration of the cancer cell.

47. (canceled)

48. The method of claim 32, wherein the cancer cell is a prostate cancer cell, a skin cancer cell, a melanoma cell, a breast cancer cell, or a lung cancer cell.

49-52. (canceled)

53. A method for treating an individual having, or suspected of having cancer, comprising administering to the individual an effective amount of a molecule capable of inhibiting expression of XAGE, a molecule capable of inhibiting expression of GAGE, and/or a molecule capable of inhibiting expression of SSX, optionally further comprising determining if one or more cancer-testis antigens are expressed in the cancer, optionally wherein the determining is performed prior to administering the molecule(s).

54. The method of claim 53, wherein the molecule capable of inhibiting expression of XAGE is or encodes a small interfering nucleic acid capable of inhibiting expression of XAGE, the molecule capable of inhibiting expression of GAGE is or encodes a small interfering nucleic acid capable of inhibiting expression of GAGE, and/or the molecule capable of inhibiting expression of SSX is or encodes a small interfering nucleic acid capable of inhibiting expression of SSX.

55. The method of claim 54, wherein the small interfering nucleic acid capable of inhibiting expression of GAGE comprises a nucleic acid sequence consisting of SEQ ID NO. 2 or SEQ ID NO. 4; and/or wherein the small interfering nucleic acid capable of inhibiting expression of XAGE comprises a nucleic acid sequence consisting of SEQ ID NO. 6 or SEQ ID NO. 8; and/or wherein the small interfering nucleic acid capable of inhibiting expression of SSX comprises a nucleic acid sequence consisting of SEQ ID NO. 12 or SEQ ID NO. 22; and/or wherein the small interfering nucleic acid capable of inhibiting expression of GAGE is a duplex having a sense strand consisting of SEQ ID NO. 1 and an antisense strand consisting of SEQ ID NO. 2; and/or wherein the small interfering nucleic acid capable of inhibiting expression of GAGE is a duplex having a sense strand consisting of SEQ ID NO. 3 and an antisense strand consisting of SEQ ID NO. 4; and/or wherein the small interfering nucleic acid capable of inhibiting expression of XAGE is a duplex having a sense strand consisting of SEQ ID NO. 5 and an antisense strand consisting of SEQ ID NO. 6; and/or wherein the small interfering nucleic acid capable of inhibiting expression of XAGE is a duplex having a sense strand consisting of SEQ ID NO. 7 and an antisense strand consisting of SEQ ID NO. 8; and/or wherein the small interfering nucleic acid capable of inhibiting expression of SSX is a duplex having a sense strand consisting of SEQ ID NO. 11 and an antisense strand consisting of SEQ ID NO. 12; and/or wherein the small interfering nucleic acid capable of inhibiting expression of SSX is a duplex having a sense strand consisting of SEQ ID NO. 21 and an antisense strand consisting of SEQ ID NO. 22.

56-63. (canceled)

64. The method of claim 54, wherein the small interfering nucleic acid capable of inhibiting expression of GAGE, XAGE or SSX is a 27-mer siRNA or a small hairpin RNA.

65-66. (canceled)

67. The method of claim 53, wherein the cancer is a prostate cancer, a skin cancer, a melanoma, a breast cancer, or a lung cancer.

68-71. (canceled)

72. The method of claim 53, wherein the individual has cancer.

73. (canceled)

74. The method of claim 53, wherein the one or more cancer-testis antigens is XAGE, GAGE, and/or SSX.

75. The method of claim 53, wherein the determining comprises obtaining a sample of the cancer from the individual.

76. The method of claim 53, wherein the molecule capable of inhibiting expression of XAGE, the molecule capable of inhibiting expression of GAGE, and/or the molecule capable of inhibiting expression of SSX is combined with a pharmaceutically acceptable carrier.


SEQUENCE LISTING (46 sequences)

SEQ ID NO: 1 - DNA, 25 bp, Artificial Sequence
  Synthetic Oligonucleotide
gaaccagcaacucaacgucaggatc

SEQ ID NO: 2 - DNA, 27 bp, Artificial Sequence
  Synthetic Oligonucleotide
gauccugacguugaguugcugguuccc

SEQ ID NO: 3 - DNA, 25 bp, Artificial Sequence
  Synthetic Oligonucleotide
guucagugaugaaguggaaccagca

SEQ ID NO: 4 - DNA, 27 bp, Artificial Sequence
  Synthetic Oligonucleotide
ugcugguuccacuucaucacugaacug

SEQ ID NO: 5 - DNA, 25 bp, Artificial Sequence
  Synthetic Oligonucleotide
gacagaagaagaucaggauacagct

SEQ ID NO: 6 - DNA, 27 bp, Artificial Sequence
  Synthetic Oligonucleotide
agcuguauccugaucuucuucugucug

SEQ ID NO: 7 - DNA, 25 bp, Artificial Sequence
  Synthetic Oligonucleotide
aagcugaaacaacgcaagcuggutt

SEQ ID NO: 8 - DNA, 27 bp, Artificial Sequence
  Synthetic Oligonucleotide
aaaccagcuugcguuguuucagcuugu

SEQ ID NO: 9 - DNA, 25 bp, Artificial Sequence
  Synthetic Oligonucleotide
ggaggacucccucucuccucuccac

SEQ ID NO: 10 - DNA, 27 bp, Artificial Sequence
  Synthetic Oligonucleotide
guggagaggagagagggaguccuccca

SEQ ID NO: 11 - DNA, 25 bp, Artificial Sequence
  Synthetic Oligonucleotide
caaggucacccucccaccuuucatg

SEQ ID NO: 12 - DNA, 27 bp, Artificial Sequence
  Synthetic Oligonucleotide
caugaaaggugggagggugaccuugaa

SEQ ID NO: 13 - DNA, 25 bp, Artificial Sequence
  Synthetic Oligonucleotide
gcuucugaaggaguucacugugucc

SEQ ID NO: 14 - DNA, 27 bp, Artificial Sequence
  Synthetic Oligonucleotide
ggacacagugaacuccuucagaagcac

SEQ ID NO: 15 - DNA, 18 bp, Artificial Sequence
  Synthetic Oligonucleotide
gaccaagacgctacgtag

SEQ ID NO: 16 - DNA, 18 bp, Artificial Sequence
  Synthetic Oligonucleotide
ccatcaggaccatcttca

SEQ ID NO: 17 - DNA, 23 bp, Artificial Sequence
  Synthetic Oligonucleotide
tcccaggagcccagtaatggaga

SEQ ID NO: 18 - DNA, 30 bp, Artificial Sequence
  Synthetic Oligonucleotide
cagcttgtcttcatttaaacttgtggttgc

SEQ ID NO: 19 - DNA, 20 bp, Artificial Sequence
  Synthetic Oligonucleotide
aaatctggcaccacaccttc

SEQ ID NO: 20 - DNA, 20 bp, Artificial Sequence
  Synthetic Oligonucleotide
cactgtgttgccgtacaggt

SEQ ID NO: 21 - DNA, 25 bp, Artificial Sequence
  Synthetic Oligonucleotide
cuuguguauccaugcaccuaccuca

SEQ ID NO: 22 - DNA, 27 bp, Artificial Sequence
  Synthetic Oligonucleotide
ugagguaggugcauggauacacaagcc

SEQ ID NO: 23 - DNA, 25 bp, Artificial Sequence
  Synthetic Oligonucleotide
ccagcuaugugaaaguccugcacca

SEQ ID NO: 24 - DNA, 27 bp, Artificial Sequence
  Synthetic Oligonucleotide
uggugcaggacuuucacauagcugguu

SEQ ID NO: 25 - DNA, 25 bp, Artificial Sequence
  Synthetic Oligonucleotide
agauuacuuuccugugauacucaag

SEQ ID NO: 26 - DNA, 27 bp, Artificial Sequence
  Synthetic Oligonucleotide
cuugaguaucacaggaaaguaaucuuu

SEQ ID NO: 27 - DNA, 25 bp, Artificial Sequence
  Synthetic Oligonucleotide
cucgaggaacguaguguucuuugca

SEQ ID NO: 28 - DNA, 27 bp, Artificial Sequence
  Synthetic Oligonucleotide
ugcaaagaacacuacguuccucgagcc

SEQ ID NO: 29 - DNA, 25 bp, Artificial Sequence
  Synthetic Oligonucleotide
cuuccucucuuucucucccuuguga

SEQ ID NO: 30 - DNA, 27 bp, Artificial Sequence
  Synthetic Oligonucleotide
ucacaagggagagaaagagaggaagga

SEQ ID NO: 31 - DNA, 25 bp, Artificial Sequence
  Synthetic Oligonucleotide
acccugaaguucaucugcaccaccg

SEQ ID NO: 32 - DNA, 27 bp, Artificial Sequence
  Synthetic Oligonucleotide
cgguggugcagaugaacuucaggguca

SEQ ID NO: 33 - DNA, 22 bp, Artificial Sequence
  Synthetic Oligonucleotide
ttaaggcacgagggaacctcac

SEQ ID NO: 34 - DNA, 18 bp, Artificial Sequence
  Synthetic Oligonucleotide
ggtatccgagtcccagaa

SEQ ID NO: 35 - DNA, 21 bp, Artificial Sequence
  Synthetic Oligonucleotide
cccaggtgctgggaagggaaa

SEQ ID NO: 36 - DNA, 20 bp, Artificial Sequence
  Synthetic Oligonucleotide
tgtggttgctcttcacctgc

SEQ ID NO: 37 - DNA, 22 bp, Artificial Sequence
  Synthetic Oligonucleotide
cggccgaaggaacctgacccag

SEQ ID NO: 38 - DNA, 23 bp, Artificial Sequence
  Synthetic Oligonucleotide
gctggaaccctcactgggttgcc

SEQ ID NO: 39 - DNA, 25 bp, Artificial Sequence
  Synthetic Oligonucleotide
aaatcgtctatgtgtatatgaagct

SEQ ID NO: 40 - DNA, 23 bp, Artificial Sequence
  Synthetic Oligonucleotide
gggtcgctgatctcttcataaac

SEQ ID NO: 41 - DNA, 23 bp, Artificial Sequence
  Synthetic Oligonucleotide
cagggctgaatggatgctgcaga

SEQ ID NO: 42 - DNA, 21 bp, Artificial Sequence
  Synthetic Oligonucleotide
gcgcctctgccctgagggagg

SEQ ID NO: 43 - DNA, 23 bp, Artificial Sequence
  Synthetic Oligonucleotide
gacgaggatcgtctcaggtcagc

SEQ ID NO: 44 - DNA, 21 bp, Artificial Sequence
  Synthetic Oligonucleotide
acatcctcaccctcaggaggg

SEQ ID NO: 45 - DNA, 19 bp, Artificial Sequence
  Synthetic Oligonucleotide
gggaatctgacggatcgga

SEQ ID NO: 46 - DNA, 19 bp, Artificial Sequence
  Synthetic Oligonucleotide
ggaatggaacgcctggaac